(19) United States
(12) Patent Application Publication
(10) Pub. No.: US 2007/0244071 A1
(43) Pub. Date: Oct. 18, 2007

(54) Treatment of proliferative diseases

(75) Inventors: Jim Dennis, Etobicoke (CA); Hyun-Joo Park Song, Oakville (CA)

Correspondence Address: HAMRE, SCHUMANN, MUELLER & LARSON, P.C., P.O. BOX 2902, MINNEAPOLIS, MN, 55402-0902, US

(73) Assignee: MOUNT SINAI HOSPITAL, TORONTO (CA)

(21) Appl. No.: 11/715,193

(22) Filed: Mar. 7, 2007

Related U.S. Application Data
(60) Provisional application No. 60/779,728, filed on Mar. 7, 2006.

(51) Int. Cl. A61K 31/695; A61K 31/655; A61K 31/24; A61K 31/35

(52) U.S. Cl. 514/63; 514/459; 514/151; 514/114; 514/534

(57) ABSTRACT

The invention relates generally to compositions comprising malonate compounds, and methods and uses of the compositions in the treatment of proliferative diseases. In particular the invention relates to a pharmaceutical composition comprising a compound of the formula I 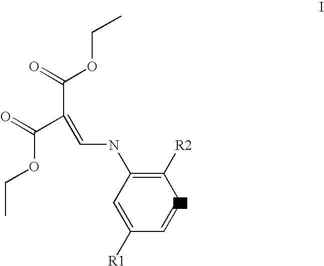 wherein * is ═C or ═O, R1 and R2 independently represent substituted or unsubstituted alkyl, alkenyl, alkynyl, alkylene, alkenylene, alkoxy, alkenyloxy, cycloalkyl, cycloalkenyl, aryl, aryloxy, arylalkoxy, aroyl, heteroaryl, heterocyclic, acyl, acyloxy, sulfonyl, sulfinyl, sulfenyl, amino, imino, azido, thiol, thioalkyl, thioalkoxy, thioaryl, nitro, ureido, cyano, halo, silyl, silyloxy, silythio, carboxyl, carbonyl, carbamoyl, carboxamide, carboxylic ester, phosphono, a substituted or unsubstituted aryl group fused to a cycloalkyl group, or a pharmaceutically acceptable salt thereof, and pharmaceutically acceptable carriers, excipients, and vehicles.

### FIELD OF THE INVENTION

The invention relates generally to compounds, compositions, and methods using the compounds and compositions to treat proliferative diseases.

### BACKGROUND OF THE INVENTION

The family of transforming growth factor-beta (TGF-β) cytokines regulate cell cycle checkpoints, cell differentiation during embryogenesis, as well as matrix production and inflammation during postnatal life (1). Ligand activation of TGF-β receptor kinases (TβR) I and II heterodimers leads to phosphorylation of Smad2/3, recruitment of Smad4 (2), and translocation of these complexes into the nucleus where they bind various transcription factor complexes and regulate gene expression (3). TGF-β signaling stimulates expression of p21 and p27, which inhibit cyclin D/Cdk4 and the G1/S cell cycle transition (4). Inactivating mutations in the type II TGF-p receptor (TβRII) and Smad4 proteins promote expansion of pre-malignant cell populations. However, in late stage cancers, TGF-β signaling promotes the invasive phenotype and cancer progression in cells that retain functional signaling machinery (5,6). Epithelial-mesenchymal transition (EMT) in carcinoma cells is accompanied by loss of E-cadherin in adhesion junctions, membrane remodeling, and cell motility (7). EMT requires a balance between TGF-β/Smad2/3, and the antagonistic Ras/Erk and PI3 kinase/Akt oncogene pathways (8,9). In this regard, TGF-β stimulates the expression of Pten phosphatase a negative regulator of PI3 kinase signaling (10). Cdk4 promotes G1/S transition, in part by phosphorylating Smad2/3 which suppresses transcription of p21 (11). Smad2/3 binds to FOXO, also a negative regulator of cell cycle, while the PI3 kinase/Akt pathway negatively regulates FOXO by blocking its transit into the nucleus (12). Therefore, chemical agents that control both Smad2/3 and Erk/PI3 kinase pathways may have novel anti-cancer activities.

In postnatal life, TGF-β/Smad suppresses Erk/p38 kinase in activated leukocytes and suppresses inflammation (13). Synthetic oleanane triterpenoids that act as suppressers of inflammation have been shown to enhance TGF-β dependent signaling (14).

### SUMMARY OF THE INVENTION

To identify enhancers of cytokine signaling, a sensitive cell-based assay was developed employing quantitative immunofluorescence imaging to quantify Erk-p and Smad2/3 levels in the cytoplasm and the nucleus. A chemical library was screened and enhancers of TGF-β-dependent Smad2/3 nuclear translocation that share a core structure of diethyl 2-(anilinomethylene)malonate (DAM) were identified. In secondary assays to characterize the biological activity of a DAM compound (e.g. DAM-1976), advantage was taken of the recently characterized Mgat5 mutation. β1,6N-acetylglucosaminyltransferase V (Mgat5) is a Golgi enzyme in the N-glycan processing pathway that modifies glycoproteins including the cytokine receptors (15). Mgat5 gene expression is up-regulated by Ras pathway activation (16,17). Galectins bind to Mgat5-modified N-glycans on EGFR and TβR at the cell surface, which delays receptor loss to constitutive endocytosis and promotes sensitivity to cytokines (15). Tumor latency is longer and metastasis is reduced in polyomavirus middle T (PyMT) transgenic Mgat5^{-/-} mice compared to PyMT transgenic Mgat5^{+/+} mice (18). The PyMT Mgat5^{-/-} mammary tumor cells display reduced sensitivity to multiple cytokines including EGF, TGF-β, IGF, PDGF and FGF (15). DAM-1976, a compound identified in the screen for modifiers of TGF-β signaling, was found to rescue sensitivity to acute EGF and TGF-β in PyMT Mgat5^{-/-}(22.9) cells, suggesting a mechanism of action that opposes membrane remodeling and endocytosis. In wild type PyMT Mgat5^{+/+}(2.6) tumors, DAM-1976 also enhances sensitivity to EGF and TGF-β, increases basal Erk and Smad2/3 activation, reduces microfilament remodeling and selectively inhibits tumor cell proliferation.

Accordingly, the invention provides a compound of the formula I which is capable of inhibiting cell proliferation: 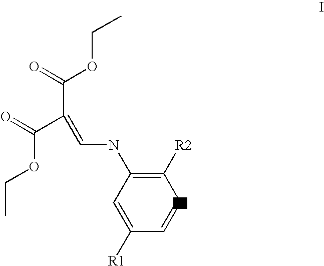
wherein * is ═C or ═O, R1 and R2 independently represent substituted or unsubstituted alkyl, alkenyl, alkynyl, alkylene, alkenylene, alkoxy, alkenyloxy, cycloalkyl, cycloalkenyl, aryl, aryloxy, arylalkoxy, aroyl, heteroaryl, heterocyclic, acyl, acyloxy, sulfonyl, sulfinyl, sulfenyl, amino, imino, azido, thiol, thioalkyl, thioalkoxy, thioaryl, nitro, ureido, cyano, halo, silyl, silyloxy, silythio, carboxyl, carbonyl, carbamoyl, carboxamide, carboxylic ester, phosphono, a substituted or unsubstituted aryl group fused to a cycloalkyl group, or a pharmaceutically acceptable salt thereof.

The present invention also relates to pharmaceutically acceptable salts and prodrugs of compounds of the formula I. Compounds of the present invention also include all stereoisomers (e.g., cis and trans isomers) and all optical isomers of compounds of the formula I (e.g., R and S enantiomers), as well as racemic, diastereomeric and other mixtures of such isomers. The invention also relates to all tautomers of compounds of the formula I.

The present invention is also directed to a pharmaceutical composition effective in treating various disorders for which a compound of the formula I has efficacy, in particular a proliferative disease. This invention further relates to pharmaceutical compositions comprising compounds of the formula I, and optionally pharmaceutically acceptable carriers, excipients, and vehicles, in particular for the treatment of a disorder for which an inhibitor of cell proliferation is efficacious or indicated. One of ordinary skill in the art will appreciate that the compounds of the formula I are useful in preventing and/or treating a diverse array of disorders or diseases.

The invention provides a composition, in particular a pharmaceutical composition, comprising a chemical enhancer of cytokine signalling, in particular a compound of the formula I, that provides beneficial effects in the treatment of a disease or disorder described herein. In an aspect the invention provides a pharmaceutical composition, comprising one or more compound of the formula I that provides beneficial effects, in particular sustained beneficial effects, following treatment. The beneficial effects provided by a compound or composition of the invention can include enhanced therapeutic effects, in particular sustained therapeutic effects.

The invention also provides a pharmaceutical composition intended for administration to a subject to provide beneficial effects, in particular sustained beneficial effects, comprising a compound of the invention, in particular a pure compound of the formula I, more particularly a substantially pure compound of the formula I, optionally together with one or more pharmaceutically acceptable carriers, excipients, or vehicles.

The invention also contemplates the use of a compound of the formula I in the manufacture of a medicament for the treatment of a disorder or disease disclosed herein.

The present invention relates to a formulation of the active agents of the formula I alone or with one or more other therapeutic agents which are to form a combination, and to co-administration methods in which the combination of agents is achieved by the simultaneous administration of the agents to be given in combination.

The invention also relates to methods for making a compound or composition of the present invention and processes and intermediates useful for making the compounds and compositions of the invention. After compositions have been prepared, they can be placed in an appropriate container and labelled for treatment of an indicated condition. For administration of a composition of the invention, such labelling would include amount, frequency, and method of administration.

The invention also provides methods to make commercially available formulations which contain a compound of the invention.

Compounds, in particular pure or substantially pure compounds of the formula I, and compositions of the invention may be administered therapeutically or prophylactically to treat diseases or disorders disclosed herein. As enhancers of cytokine signaling the compounds of the formula I may be useful in the treatment, reduction of the risk of, or prevention of diseases and disorders which benefit from enhancing cytokine signaling, in particular in the treatment of proliferative diseases.

The present invention relates to a method of eliciting an enhanced cytokine signaling response in a subject in need thereof, comprising administering to the subject a therapeutically effective amount of a compound of the formula I or a composition of the invention.

In another aspect, the invention provides a method of enhancing TGF-β dependent Smad2/3 nuclear translocation in a subject in need thereof by administering a compound or composition of the invention.

In another aspect, the invention provides a method of inhibiting microfilament remodeling and enhancing sensitivity to cytokines by prolonging the trafficking and activation of signaling intermediates in a subject in need thereof by administering a compound or composition of the invention.

The present invention also relates to use of a compound of the invention for eliciting an enhanced cytokine signaling response in a subject as well as use of a compound of the invention to prepare a medicament for eliciting an enhanced cytokine signaling response in a subject.

Other features and advantages of the present invention will become apparent from the following detailed description. It should be understood, however, that the detailed description, and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

FIG. 1. Identification of compounds that enhance TGF-β signaling (A) Time course of Smad2/3 nuclear translocation in MNuMG mammary epithelial cells following the addition of TGF-β1. Cells were stained with anti-Smad2/3 antibodies, followed by a fluorescent-labeled secondary antibody as described in the Example. The nuclear and cytoplasmic staining intensity was determined individually for 100 cells per well, and nuclear minus cytoplasmic was used to represent the change in activation following addition of cytokine. The S.E. of the mean (n=100) was generally<4% for each assay point. (B) Sample of data from the primary compound screen for modifiers of Smad2/3 nuclear translocation in MNuMG cells. Each point represents the mean nuclear translocation of Smad2/3 in a well. (C) Dose responses for compound enhancement of TGF-β-mediated Smad2/3 nuclear translocation in NMuMG cells. D₅₀ values are listed in the box. (D) Time course of TGF-β dependent Smad2/3 nuclear translocation following compound sensitization of NMuMG cells. Cells were pre-treated with DAM-1976 for 2 h prior to addition of 50 pM of TGF-β and Smad2/3 nuclear translocation was measured at the indicated times. (E) Immunofluorescence images of NMuMG cells stained with anti-phospho-Smad2/3 antibodies. Cells were either untreated or pre-treated with 20 μM DAM-1976 for 2 h, then stimulated with buffer or 50 pM TGF-β for 45 min.

FIG. 2. DAM-1976 rescues Mgat5^{-/-} and sensitizes Mgat5^{+/+} mammary carcinoma cells to EGF and TGF-β. Tonic and acute activation of Smad2/3 and Erk-p in (A,B) Mgat5^{+/+}(2.6) and (C,D) Mgat5^{-/-}(22.9) cells treated with DAM-1976 for 2 h prior to addition of cytokine or no addition. For acute stimulation with TGF-β (50 pM) or EGF (100 ng/ml), measurements of nuclear Smad2/3 at 45 min and nuclear Erk-p at 5 min, respectively, are shown. (E) Mgat5^{+/+}(2.6) and Mgat5^{-/-}(22.9) cells were cultured with and without 5 μM DAM-1976 for 48 h in DMEM+10% FCS, and stained for E-cadherin (green) and actin microfilaments with rhodamine-phalloidin (red).

FIG. 3. DAM-1976 delays EGFR internalization and prolongs Erk-p activation. (A) Nuclear translocation of Erk1/2 in Mgat5^{+/+}(2.6) cells pretreated with DAM-1976 for 2 h and stimulated with 100 ng/ml of EGF. (B) Phosphorylation of the EGFR and Erk1/2 following stimulation with 100 ng/ml of EGF for 10 min with and without DAM-1976 (5 μM) pretreatment for 2 h. (C) Mgat5^{+/+}(2.6) cells were cultured in the presence or absence of 10 μM DAM-1976 for 24 h, then stimulated with either buffer or EGF for 20 min. Surface proteins were biotinylated with sulfosuccinimidyl-6-(biotinamido) hexanoate (Sulfo-NHS-LC-biotin), captured on streptavidin-agarose beads, separated by SDS-PAGE and probed with anti-EGFR antibodies. (D) Surface EGFR biotinylation at times after stimulation of Mgat5^{+/+}(2.6) cells with EGF. Densitometry of Western blots following streptavidin-agarose pull-downs and probing with anti-EGFR. The data normalized to time 0 untreated.

FIG. 4. DAM-1976 inhibits microfilament turnover, cell spreading and tumor cell growth. (A) Mgat5^{+/+}(2.6) and Mgat5^{-/-}(22.9) cells were treated with 100 ng/ml of LatA, fixed at times thereafter, stained with rhodamine-phalloidin and basolateral micofilament density in a 0.5μ cytoplasmic ring was measured. (B) Mgat5^{+/+}(2.6) and Mgat5^{-/-}(22.9) cell plated on 0.5 μg/ml fibronectin were pre-treated with and without 10 μM DAM-1976 for 18 h, then with and without 100 ng/ml of LatA for 20 min. Total rhodamine-phalloidin intensity per cell was measured with GE Incell-1000 imager, and the results are the mean±S.D. of 3 replicate wells. (C) Mgat5^{+/+}(2.6) were cultured with and without 10 μM DAM-1976 for 18 h in DMEM+10% FBS then treated with 100 ng/ml of LatA, and cell area was determined at times thereafter. (D) Effects of pretreatment with DAM-1976 alone, on cell spreading in serum-free medium 4 h after plating into wells coated with 0.5 μg/ml of fibronectin. Cells were pretreated with DAM-1976 for 18 h at the indicated concentrations. (E) Relative cell number following 48 h of growth in DMEM+10% FBS supplemented with DAM-1976. (F) Cell cycle profile of Mgat5^{+/+}(2.6) cells treated with DAM-1976 for 24 h in DMEM+10% FBS.

FIG. 5. DAM-1976 Inhibits tumor cell growth and stimulates basal Erk-p and Smad2/3. Effects of (A) TGF-β and (B) DAM-1976 on cell number after 72 h of growth in DMEM, 1% FBS measured by the AlamarBlue homogenous assay. Steady-state levels of (C,D) nuclear phospho-Smad2/3 (E,F) nuclear Erk-p in cells after 72 h of growth in MEM, 1% FBS. DAM-1976 induced a greater loss of substratum attachment and death of Mgat5^{+/+}(2.6) carcinoma cells compared to MvLu and DR26 cells; hence the absence of Erk-p and Smad2/3 data at >10 μM. The results are representative of 3 experiments.

### DETAILED DESCRIPTION OF EMBODIMENTS

As used throughout the disclosure, the following terms, unless otherwise indicated, shall be understood to have the following meanings.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made. Further, it is to be understood that “a,” “an,” and “the” include plural referents unless the content clearly dictates otherwise. Thus, for example, reference to a composition comprising “a compound” includes a mixture of two or more compounds. “Compound(s)” or “compound(s) of the invention” refers to any compound encompassed by the general formula I disclosed herein, including, without limitation, pharmaceutically acceptable salts, isomers, tautomers, solvates, hydrates, derivatives and prodrugs thereof.

Compounds of the invention can exist in tautomeric, geometric or stereoisomeric forms. Pharmaceutically acceptable salts of such tautomeric, geometric or stereoisomeric forms are also included within the invention.

Compounds disclosed herein may contain one or more chiral centers and/or double bonds and therefore, may exist as stereoisomers, such as double-bond isomers (i.e., geometric isomers), enantiomers or diastereomers. Accordingly, a compound of the formula I encompasses all possible enantiomers and stereoisomers including the stereoisomerically pure form (e.g., geometrically pure, enantiomerically pure or diastereomerically pure) and enantiomeric and stereoisomeric mixtures. In particular, compounds, including cis- and trans-geometric isomers, E- and Z-geometric isomers, R- and S-enantiomers, diastereomers, d-isomers, 1-isomers, the racemic mixtures thereof and other mixtures thereof, fall within the scope of the invention. Enantiomeric and stereoisomeric mixtures can be resolved into their component enantiomers or stereoisomers using separation techniques or chiral synthesis techniques well known to the skilled artisan.

Compounds of the formula I disclosed herein may exist in several tautomeric forms including the enol form, the keto form and mixtures thereof, and therefore the compounds of the invention encompass all possible tautomeric forms of the illustrated compounds.

The compounds disclosed herein also include isotopically labeled compounds of the formula I wherein one or more atoms have an atomic mass different from the atomic mass conventionally found in nature. Examples of isotopes that may be incorporated into the compounds of the invention include, without limitation, ²H, ³H, ¹³C, ¹⁴C, ¹⁵N, ¹⁸O, ¹⁷O, etc.

Compounds of the formula I of the invention may exist in unsolvated and solvated forms, including hydrated forms and as N-oxides. Certain compounds of the invention may exist in various crystalline or amorphous forms. All physical forms of a compound of the formula I are equivalent for the uses contemplated herein and are intended to be within the scope of the present invention.

The term “pure” in general means better than 90%, 92%, 95%, 97%, 98% or 99% pure, and “substantially pure” means a compound synthesized such that the compound, as made as available for consideration into a composition or therapeutic dosage of the invention, has only those impurities that can not readily nor reasonably be removed by conventional purification processes.

The compounds disclosed herein also include “pharmaceutically acceptable salt(s)”. By pharmaceutically acceptable salts is meant those salts which are suitable for use in contact with the tissues of a subject or patient without undue toxicity, irritation, allergic response and the like, and are commensurate with a reasonable benefit/risk ratio. Pharmaceutically acceptable salts are well known in the art and are described for example, in S. M. Berge, et al., J. Pharmaceutical Sciences, 1977, 66:1.

The term “pharmaceutically acceptable salt(s)” includes salts of acidic or basic groups which may be present in the compounds of the present invention. In particular the present invention provides pharmaceutically acceptable acid addition salts of compounds of the formula I. The acids which are used to prepare the pharmaceutically acceptable acid addition salts of the compounds of the Formula I are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, para-toluenesulfonate and pamoate [i.e., 1,1′-methylene-bis-(2-hydroxy-3-naphthoate)] salts.

In other aspects, the invention relates to the pharmaceutically acceptable base addition salts of formula I. Chemical bases that may be used as reagents to prepare pharmaceutically acceptable base salts of compounds of formula I that are acidic in nature are those that form non-toxic base salts with such compounds. Suitable non-toxic base salts include, without limitation, those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine (meglumine), and the lower alkanolammonium and other base salts of pharmaceutically acceptable organic amines.

As used herein, the term “prodrug” refers to a compound of Formula I, or a compound of the Formula I comprising structural modifications thereto, such that in vivo the prodrug is converted, for example, by hydrolytic, oxidative, reductive, or enzymatic cleavage into a parent compound. The term includes bioreversible derivatives of drug molecules used to overcome some barriers to the utility of the parent drug molecule. Examples of barriers include, without limitation, solubility, permeability, stability, presystemic metabolism and targeting limitations (J. Stella, “Prodrugs as therapeutics”, Expert Opin. Ther. Patents, 14(3), 277-280, 2004). Prodrugs may be, for example, metabolically labile mono- or di-ester derivatives of a parent compound having a carboxylic acid group. Thus, in an aspect, the present invention includes any such prodrugs, such as metabolically labile ester or diester compounds of the formula I or derivatives thereof. In all cases, the use of the compounds described herein as prodrugs is contemplated, and often is preferred, and thus, the prodrugs of all of the compounds employed are encompassed in the names of the compounds herein.

Accordingly, in aspects of the invention, R1 and/or R2 or parts thereof may be cleavable by esterases and/or other enzymes that generate a hydroxyl group. In other aspects, R1 and/or R2 or parts thereof are cleavable by chemical hydrolysis. “Alkyl”, either alone or within other terms such as “thioalkyl” and “arylalkyl” refers to a monovalent, saturated hydrocarbon radical which may be a straight chain (i.e. linear) or a branched chain. In certain aspects of the invention an alkyl radical comprises from about 1 to 20 carbon atoms, preferably from about 1 to 10 or 3 to 8, more preferably about 3 to 6, 1 to 4, or 1 to 3 carbon atoms. Examples of alkyl radicals include methyl, ethyl, n-propyl, n-butyl, n-pentyl, n-hexyl, isopropyl, isobutyl, isopentyl, amyl, sec-butyl, tert-butyl, tert-pentyl, n-heptyl, n-octyl, n-nonyl, n-decyl, n-dodecyl, n-tetradecyl, n-hexadecyl, n-octadecyl, and the like, along with branched variations thereof. In certain embodiments of the invention an alkyl radical is a C₁-C₆ alkyl comprising or selected from the group consisting of methyl, ethyl, n-propyl, n-butyl, n-pentyl, n-hexyl, isopropyl, isobutyl, isopentyl, amyl, sec-butyl, tert-butyl, tert-pentyl, and n-hexyl. An alkyl radical may be optionally substituted with substituents at positions that do not significantly interfere with the preparation of compounds of the formula I and that do not significantly reduce the efficacy of the compounds. An alkyl radical may be optionally substituted with groups as defined herein. In certain aspects, an alkyl radical is substituted with one to five substituents independently selected from the group consisting of halo, lower alkoxy, hydroxy, cyano, nitro, thio, amino, substituted amino, carboxyl, and substituted carboxyl.

The term “cycloalkyl” as used herein refers to radicals having from about 3 to 15 carbon atoms and containing one, two, three, or four rings wherein the rings may be attached in a pendant manner or may be fused. In aspects of the invention the cycloalkyl radicals are “lower cycloalkyl” radicals having from about 3 to 8 carbon atoms, in particular cyclopropyl, cyclobutyl, cyclopentyl, cyclohexyl and cycloheptyl. A cycloalkyl radical may be optionally substituted with groups as disclosed herein.

The term “alkenyl” as used herein refers to an unsaturated, acyclic branched or straight-chain hydrocarbon radical comprising at least one double bond. An alkenyl radical may contain from about 2 to 10 carbon atoms, in particular from about 3 to 8 carbon atoms and more particularly about 3 to 6 carbon atoms. Examples of alkenyl radicals include ethenyl, propenyl such as prop-1-en-1-yl, prop-1-en-2-yl, prop-2-en-1-yl (allyl), prop-2-en-2-yl, buten-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, hexen-1-yl, 3-hydroxyhexen-1-yl, hepten-1-yl, and octen-1-yl, and the like. An alkenyl radical may be optionally substituted similar to alkyl. The term “cycloalkenyl” as used herein refers to cyclic alkenyl groups including without limitation cyclobutenyl, cyclopentenyl, cyclohexenyl and cycloheptenyl. A cycloalkenyl radical may be optionally substituted with groups as disclosed herein.

The term “alkynyl” as used herein refers to an unsaturated, branched or straight-chain hydrocarbon radical comprising one or more triple bonds. Alkynyl radicals may contain about 1 to 20, 1 to 15, or 2-10 carbon atoms, preferably about 3 to 8 carbon atoms and more preferably having about 3 to 6 or 2 to 4 carbon atoms. Examples of alkynyl radicals include ethynyl, such as prop-1-yn-1-yl, prop-2-yn-1-yl butynyls such as but-1-yn-1-yl, but-1-yn-3-yl, but-3-yn-1-yl, pentynyls such as pentyn-1-yl, pentyn-2-yl, 4-methoxypentyn-2-yl, 3-methylbutyn-1-yl, hexynyls such as hexyn-1-yl, hexyn-2-yl, hexyn-3-yl, and 3,3-dimethylbutyn-1-yl radicals and the like. This radical may be optionally substituted similar to alkyl. The term “cycloalkynyl” refers to cyclic alkynyl groups.

The term “alkylene” as used herein refers to a linear or branched radical having from about 1 to 10 carbon atoms and having attachment points for two or more covalent bonds. Examples of alkylene radicals are methylene, ethylene, ethylidene, methylethylene, and isopropylidene.

The term “alkenylene” as used herein refers to a linear or branched radical having from about 2 to 10 carbon atoms, at least one double bond, and having attachment points for two or more covalent bonds. Examples of alkenylene radicals are 1,1-vinylidene (CH₂═C), 1,2-vinylidene (—CH═CH—), and 1,4-butadienyl (—CH═CH—CH═CH—).

The term “halo” as used herein refers to halogens such as fluorine, chlorine, bromine or iodine atoms.

The term “cyano” as used herein refers to a carbon radical having three or four covalent bonds shared by a nitrogen atom, in particular —CN.

The term “alkoxy” as used herein refers to a linear or branched oxy-containing radical having an alkyl portion of one to about ten carbon atoms, such as a methoxy radical, which may be substituted. Particular alkoxy radicals are “lower alkoxy” radicals having about 1 to 6, 1 to 4, or 1 to 3 carbon atoms. The term “alkoxy” having about 1-6 carbon atoms means a C₁-C₆ alkyl-O- group or radical wherein C₁-C₆ alkyl has the meaning as defined above. Examples of alkoxy radicals include without limitation methoxy, ethoxy, propoxy, butoxy, isopropoxy and tert-butoxy alkyls. An “alkoxy” radical may optionally be further substituted with one or more substitutents disclosed herein including alkyl atoms to provide “alkoxyalkyl” radicals; and halo atoms, such as fluoro, chloro or bromo, to provide “haloalkoxy” radicals (e.g. fluoromethoxy, chloromethoxy, trifluoromethoxy, difluoromethoxy, trifluoroethoxy, fluoroethoxy, tetrafluoroethoxy, pentafluoroethoxy, and fluoropropox) and “haloalkoxyalkyl” radicals (e.g. fluoromethoxymethyl, chloromethoxyethyl, trifluoromethoxymethyl, difluoromethoxyethyl, and trifluoroethoxymethyl).

The term “alkenyloxy” as used herein refers to linear or branched oxy-containing radicals having an alkenyl portion of about 2 to 10 ten carbon atoms, such as ethenyloxy or propenyloxy radical. Particular alkenyloxy radicals are “lower alkenyloxy” radicals having about 2 to 6 carbon atoms. Examples of alkenyloxy radicals include ethenyloxy, propenyloxy, butenyloxy, and isopropenyloxy alkyls. An “alkenyloxy” radical may be substituted with one or more substitutents disclosed herein including halo atoms, such as fluoro, chloro or bromo, to provide “haloalkenyloxy” radicals (e.g. trifluoroethenyloxy, fluoroethenyloxy, difluoroethenyloxy, and fluoropropenyloxy).

The term “cycloalkoxy” refers to cycloalkyl radicals attached to an oxy radical. Examples of cycloalkoxy radicals includes cyclohexoxy and cyclopentoxy. A cycloalkoxy radical may be optionally substituted with groups as disclosed herein.

The term “aryl”, alone or in combination, refers to a carbocyclic aromatic system containing one, two or three rings wherein such rings may be attached together in a pendant manner or may be fused. The term “fused” means that a second ring is present (i.e, attached or formed) by having two adjacent atoms in common or shared with the first ring. The term “aryl” includes without limitation phenyl, naphthyl, anthraceneyl, 1,2-dihydronaphthyl, tetrahydronaphthyl, indanyl, indenyl, fluorenyl, biphenyl, and the like. An aryl radical may be optionally subsitituted with groups as disclosed herein.

The term “aryloxy” refers to aryl radicals, as defined above, attached to an oxygen atom. Exemplary aryloxy groups include napthyloxy, quinolyloxy, isoquinolizinyloxy, and the like.

The term “arylalkoxy,” as used herein, refers to an aryl group attached to an alkoxy group. Examples of arylalkoxy include, but are not limited to, 2-phenylethoxy, 3-naphth-2-ylpropoxy, and 5-phenylpentyloxy.

The term “aroyl” as used herein refers to aryl radicals attached to a carbonyl radical, including without limitation benzoyl and toluoyl. An aroyl radical may be optionally substituted with groups as disclosed herein.

The term “heteroaryl” as used herein refers to fully unsaturated heteroatom-containing ring-shaped aromatic radicals having from 5 to 15 ring members selected from carbon, nitrogen, sulfur and oxygen, wherein at least one ring atom is a heteroatom. A heteroaryl radical may contain one, two or three rings and the rings may be attached in a pendant manner or may be fused. Examples of “heteroaryl” radicals, include without limitation, an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyrimidyl, pyrazinyl, pyridazinyl, triazolyl, tetrazolyl and the like; an unsaturated condensed heterocyclic group containing 1 to 5 nitrogen atoms, in particular, indolyl, isoindolyl, indolizinyl, benzimidazolyl, quinolyl, isoquinolyl, indazolyl, benzotriazolyl, tetrazolopyridazinyl and the like; an unsaturated 3 to 6-membered heteromonocyclic group containing an oxygen atom, in particular, 2-furyl, 3-furyl, and the like; an unsaturated 5 to 6-membered heteromonocyclic group containing a sulfur atom, in particular, 2-thienyl, 3-thienyl, and the like; unsaturated 5 to 6-membered heteromonocyclic group containing 1 to 2 oxygen atoms and 1 to 3 nitrogen atoms, in particular, oxazolyl, isoxazolyl, and oxadiazolyl; an unsaturated condensed heterocyclic group containing 1 to 2 oxygen atoms and 1 to 3 nitrogen atoms, in particular benzoxazolyl, benzoxadiazolyl and the like; an unsaturated 5 to 6-membered heteromonocyclic group containing 1 to 2 sulfur atoms and 1 to 3 nitrogen atoms, for example, thiazolyl, thiadiazolyl and the like; an unsaturated condensed heterocyclic group containing 1 to 2 sulfur atoms and 1 to 3 nitrogen atoms such as benzothiazolyl, benzothiadiazolyl and the like. The term also includes radicals where heterocyclic radicals are fused with aryl radicals, in particular bicyclic radicals such as benzofuran, benzothiophene, and the like. A heteroaryl radical may be optionally substituted with groups as disclosed herein.

The term “heterocyclic” refers to saturated and partially saturated heteroatom-containing ring-shaped radicals having from about 5 to 15 ring members selected from carbon, nitrogen, sulfur and oxygen, wherein at least one ring atom is a heteroatom. A heterocylic radical may contain one, two or three rings wherein such rings may be attached in a pendant manner or may be fused. Examples of saturated heterocyclic radicals include without limitation a saturated 3 to 6-membered heteromonocylic group containing 1 to 4 nitrogen atoms [e.g. pyrrolidinyl, imidazolidinyl, piperidino, and piperazinyl]; a saturated 3 to 6-membered heteromonocyclic group containing 1 to 2 oxygen atoms and 1 to 3 nitrogen atoms [e.g. morpholinyl]; and, a saturated 3 to 6-membered heteromonocyclic group containing 1 to 2 sulfur atoms and 1 to 3 nitrogen atoms [e.g., thiazolidinyl] etc. Examples of partially saturated heterocyclyl radicals include without limitation dihydrothiophene, dihydropyran, dihydrofuran and dihydrothiazole. Illustrative heterocyclic radicals include without limitation 2-pyrrolinyl, 3-pyrrolinyl, pyrrolindinyl, 1,3-dioxolanyl, 2H-pyranyl, 4H-pyranyl, piperidinyl, 1,4-dioxanyl, morpholinyl, 1,4-dithianyl, thiomorpholinyl, and the like.

The term “sulfonyl”, used alone or linked to other terms such as alkylsulfonyl or arylsulfonyl, refers to the divalent radicals —SO₂—.

The term “sulfinyl”, used alone or linked to other terms such as alkylsulfinyl (i.e. S(O)-alkyl) or arylsulfinyl, refers to the divalent radicals —S(O)—.

The term “amino”, alone or in combination, refers to a radical where a nitrogen atom (N) is bonded to three substituents being any combination of hydrogen, alkyl, alkenyl, alkynyl, cycloalkyl, or aryl, with the general chemical formula —NR5R6 where R5 and R6 can be any combination of hydrogen, alkyl, alkoxy, alkenyl, alkynyl, cycloalkyl, or aryl: Optionally one substituent on the nitrogen atom may be a hydroxyl group (—OH) to provide an amine known as a hydroxylamine. Illustrative examples of amino groups are amino (—NH₂), alkylamine such as methylamine, ethylamine, dimethylamine, 2-propylamine, butylamine, isobutylamine, cycloalkylamine including cyclopropylamine, benzylamine, allylamine, hydroxylamine, cyclohexylamino, piperidine and benzylamino.

The term “thioalkyl”, alone or in combination, refers to a chemical functional group where a sulfur atom (S) is bonded to an alkyl, which may be substituted. Examples of thioalkyl groups are thiomethyl, thioethyl, and thiopropyl.

The term “thioalkoxy”, alone or in combination, refers to a chemical functional group where a sulfur atom (S) is bonded to an alkoxy group with the general chemical formula —SR7 where R7 is an alkoxy group which may be substituted. In aspects of the invention a “thioalkoxy group” has 1-6 carbon atoms and refers to a S—(O)—C₁-C₆ alkyl group or radical wherein C₁-C₆ alkyl have the meaning as defined above. Examples of a straight or branched thioalkoxy group or radical having from 1 to 6 carbon atoms, also known as a C₁-C₆ thioalkoxy, include thiomethoxy and thioethoxy.

The term, “thioaryl”, alone or in combination, refers to a chemical functional group where a sulfur atom (S) is bonded to an aryl group with the general chemical formula —SR8 where R8 is an aryl group which may be substituted. Examples of thioaryl groups and substituted thioaryl groups are thiophenyl, para-chlorothiophenyl, thiobenzyl, 4-methoxy-thiophenyl, 4-nitro-thiophenyl, and para-nitrothiobenzyl.

The term “carbonyl” refers to a carbon radical having two of the four covalent bonds shared with an oxygen atom.

The term “carboxy”, alone or in combination, refers to a hydroxyl radical attached to one of two unshared bonds in a carbonyl group.

The term “carboxyl”, alone or in combination, refers to —C(O)OR9- wherein R9 is hydrogen, an organic cation or an inorganic cation.

The term “carboxamide”, alone or in combination, refers to amino, monoalkylamino, dialkylamino, monocycloalkylamino, alkylcycloalkylamino, and dicycloalkylamino radicals, attached to one of two unshared bonds in a carbonyl group.

The term “carboxylic ester”, alone or in combination, refers to —C(O)OR10- where R10, for example, is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted.

The term “acyl”, alone or in combination, means a carbonyl or thiocarbonyl group bonded to a radical selected from, for example, optionally substituted, hydrido, alkyl (e.g. haloalkyl), alkenyl, alkynyl, alkoxy (“acyloxy” including substituted acyloxy such as alkoxyalkyl and haloalkoxy), aryl, heterocyclyl, heteroaryl, sulfinyl (e.g. alkylsulfinylalkyl), sulfonyl (e.g. alkylsulfonylalkyl), cycloalkyl, cycloalkenyl, thioalkyl, thioaryl, amino (e.g alkylamino or dialkylamino), and aralkoxy. Examples of “acyl” radicals are formyl, acetyl, benzoyl, trifluoroacetyl, phthaloyl, malonyl, nicotinyl, and the like.

The term “acyloxy” means an “acyl” radical univalently bonded to divalent oxygen and includes without limitation acetyloxy, butyryloxy, iso-valeryloxy, phenylacetyloxy, benzoyloxy, p-methoxybenzoyloxy.

The term “phosphono” refers to a pentavalent phosphorus attached with two covalent bonds to an oxygen radical.

The term “substituted,” as used herein, means that any one or more hydrogens on the designated atom is replaced with a selection from a group disclosed herein, provided that the designated atom's normal valency is not exceeded, and that the substitution results in a stable compound. Combinations of substituents and/or radicals are generally permissible only if such combinations result in stable compounds. “Stable compound” or “stable structure” refers to a compound that is sufficiently robust to survive isolation to a useful degree of purity from a reaction mixture, and formulation into an efficacious therapeutic agent.

A radical in a compound of the formula I may be substituted with one or more substituents apparent to a person skilled in the art including without limitation hydroxy, halo (i.e., fluoro, bromo, chloro, or iodo), nitro, azido, cyano, alkyl, alkenyl, alkynyl, alkanoyl, alkylene, alkenylene, hydroxyalkyl, haloalkyl, haloalkylene, haloalkenyl, alkoxy, alkenyloxy, alkenyloxyalkyl, alkoxyalkyl, aryl, haloalkoxy, haloalkenyloxy, heterocyclyl, heteroaryl, sulfonyl, alkylsulfonyl, sulfinyl, alkylsulfinyl, aralkyl, heteroaralkyl, cycloalkyl, cycloalkenyl, cycloalkoxy, cycloalkenyloxy, amino, oxy, thio, thioalkyl, alkylamino, arylamino, arylsulfonyl, heteroarylsulfinyl, heteroarylsulfonyl, heteroarylamino, heteroaryloxy, heteroaryloxylalkyl, aryloxy, aroyl, aralkanoyl, aralkoxy, aryloxyalkyl, haloaryloxyalkyl, heteroaroyl, heteroaralkanoyl, heteroaralkoxy, heteroaralkoxyalkyl; thioaryl, arylthioalkyl, alkoxyalkyl, acyl groups, aroyl, heteroaryl, aryloxy, carbonyl, alkoxy carbonyl, alkylcarbonyl, nitrate, nitrite, thionitrate, thionitrite, carboxamide, carboxyl, and carboxylic ester.

The terms “subject” and “patient” used interchangeably herein, refer to an animal including a warm-blooded animal such as a mammal, which is afflicted with or suspected of having or being pre-disposed to a disorder discussed herein. Mammal includes without limitation any members of the Mammalia. In general, the terms refer to a human. The terms also include domestic animals bred for food, sport, or as pets, including horses, cows, sheep, poultry, fish, pigs, cats, dogs, and zoo animals, goats, apes (e.g. gorilla or chimpanzee), and rodents such as rats and mice. The methods herein for use on subjects/patients contemplate prophylactic as well as curative use. Typical subjects for treatment include persons susceptible to, suffering from or that have suffered a disorder described herein. In certain aspects of the invention, the subject is refractory or non-responsive to current treatments for a disorder.

The term “treating” refers to reversing, alleviating, or inhibiting the progress, severity, and/or duration of a disorder, or one or more symptoms of such disorder, to which such term applies. Depending on the condition of the patient, the term also refers to preventing a disorder, and includes preventing the onset of a disorder, or preventing the symptoms associated with a disorder. The term also refers to reducing the severity of a disorder or symptoms associated with such disorder prior to affliction with the disorder. Such prevention or reduction of the severity of a disorder prior to affliction refers to administration of a compound or composition of the present invention to a subject that is not at the time of administration afflicted with the disease or disorder. “Preventing” also refers to preventing the recurrence of a disease or disorder or of one or more symptoms associated with such disorder. The terms “treatment” and “therapeutically,” refer to the act of treating, as “treating” is defined above.

The term “pharmaceutically acceptable carrier, excipient, or vehicle” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbants that may be needed in order to prepare a particular composition. Examples of carriers excipients, or vehicles include but are not limited to saline, buffered saline, dextrose, water, glycerol, ethanol, and combinations thereof. The use of such media and agents for an active substance is well known in the art.

A “beneficial effect” refers to an effect of a compound of the formula I or composition thereof including favorable pharmacological and/or therapeutic effects, and improved pharmacokinetic properties and biological activity. A beneficial effect may be one or more of the following: chemical enhancement of cytokine signaling; enhanced TGF-β dependent Smad2/3 nuclear translocation; targeting of cells with a D₅₀ value of about 1-4 μM; inhibition of microfilament remodelling; enhanced sensitivity to cytokines by prolonging the trafficking and activation of signaling intermediates; stabilization of microfilaments; inhibition of microfilament remodeling; increased G2/M and S phase cells; delayed or slowed cell cycle progression; slower endocytosis of EGFR; increased sensitivity to EGF and TGF-β cytokines; enhanced or restored responsiveness to EGF and/or TGF-β; increased responsiveness to anabolic cytokines; promotion of stem cell survival and maintenance; increased cytokine residency at the cell surface; increased basal nuclear Erk-p and Smad2/3; growth suppression; reduced metastasis and/or, modulation of receptor trafficking both before and after ligand binding.

A beneficial effect may be a statistically significant effect in terms of statistical analysis of an effect of a compound of the invention versus the effects without the compound. A “statistically significant” effect represents an effect that is higher or lower than a standard. In embodiments of the invention, the difference may be 1.5, 2, 3, 4, 5, or 6 times higher or lower compared with the effect obtained without a compound of the invention. “Therapeutically effective amount” relates to the amount or dose of an active compound or composition of the invention that will lead to one or more desired beneficial effects. A therapeutically effective amount of a substance can vary according to factors such as the disease state, age, sex, and weight of the subject, and the ability of the substance to elicit a desired response in the subject. Dosage regime may be adjusted to provide the optimum therapeutic response. For example, several divided doses may be administered daily or the dose may be proportionally reduced as indicated by the exigencies of the therapeutic situation.

As used herein, the terms “disorder” and “disease” are used interchangeably to refer to a condition in a subject. “Disorder” and “disease” include but are not limited to diseases or disorders where enhancers of cytokine signaling are efficacious or indicated, in particular conditions associated with abnormal cell growth and/or angiogenesis. In aspects of the invention the disorder or disease is a proliferative disease or immunodeficiency disease. Certain conditions may be characterized as more than one disease or disorder.

“Proliferative disease” means a class of diverse disorders and diseases characterized by a lack of control or poorly controlled cell division or proliferation. Proliferative diseases include disorders associated with an overgrowth of connective tissues, such as various fibrotic conditions, including scleroderma, arthritis, juvenile arthritis, gouty arthritis, and liver cirrhosis, and conditions such as restenosis, arteriosclerosis, and proliferative diabetic retinopathy. Proliferative disorders also refers to neoplastic disorders including without limitation, anal cancer, bile duct cancer, colon cancer, esophageal cancer, gallbladder cancer, pancreatic cancer, small intestine cancer, stomach cancer, osteosarcoma, ovarian epithelial cancer, gestational trophoblastic tumor, uterine sarcoma, vaginal cancer, vulvar cancer, ovarian germ cell tumor, soft tissue sarcoma, hematopoietic malignancies including acute lymphoblastic leukemia, acute myeloid leukemia, and chronic myelogenous leukemia, lung cancer, small cell lung cancer, malignant mesothelioma, malignant thymoma, hypopharyngeal cancer, laryngeal cancer, nasopharyngeal cancer, oropharyngeal cancer, parathyroid cancer, salivary gland cancer, brain tumor, glioma, cerebellar astrocytoma, cerebral astrocytoma, ependymoma, medulloblastoma, adrenocortical carcinoma, pituitary tumor, islet cell carcinoma, bladder cancer, kidney cancer, penile cancer, Wilm's tumor, AIDS-related lymphoma, cutaneous T-cell lymphoma, Hodgkin's lymphoma, Ewing's sarcoma, skin cancer, hemangiomas of infancy and childhood, mycosis funoides, hairy cell leukemia, Kaposi's sarcoma, non-hodgkin's lymphoma, multiple myeloma, basal cell carcinoma, malignant melanoma, colorectal cancer, non-small cell lung carcinoma, bladder cancer, pancreatic carcinoma, renal cell carcinoma, neuroblastoma, bladder cancer, breast cancer, cervical cancer, liver cancer, sarcomas, thyroid cancer, endometrial cancer, uterine cancer, multiple myeloma, testicular cancer, retinoblastoma, colorectal cancer, oral cancer, rectal cancer, and prostate cancer. The singular form “proliferative disease” includes any one or more diseases selected from the class of proliferative diseases, and includes any compound or complex disease state wherein a component of the disease state includes a disease selected from the class of proliferative diseases. The term also includes proliferative disorders refractory to treatment with other chemotherapeutics or that are refractory to treatment with other chemotherapeutics due to multidrug resistance.

In aspects of the invention, the proliferative disease is cancer, restenosis, psoriasis, atherosclerosis, or endometriosis.

In aspects of the invention the proliferative disease is a cell proliferative disorder including cancer, skeletal disorders, angiogenic or blood vessel proliferative disorders, fibrotic disorders, and mesangial cell proliferative disorders.

In aspects of the invention, the proliferative disease is a fibrotic proliferative disorder (i.e., abnormal formation of extracellular matrices) including without limitation atherosclerosis, hepatic cirrhosis, and mesangial cell proliferative disorders (including human renal diseases, such as glomerulonephritis, diabetic nephropathy, malignant nephrosclerosis, thrombotic microangiopathy syndromes, transplant rejection and glomerulopathies).

In aspects of the invention the disorder or disease is a condition associated with abnormal cell growth. In an embodiment, the abnormal cell growth is cancer. The term “cancer” includes without limitation the following cancers: breast, ovary, cervix, prostate, testis, esophagus, glioblastoma, neuroblastoma, stomach, skin, keratoacanthoma, lung, epidermoid carcinoma, bone, pancreas, large cell carcinoma, vagina, vulva, Hodgkin's Disease, thyroid gland, adenocarcinoma, adrenal gland, prostate, chronic or acute leukemia, neoplasms of the central nervous system, primary CNS lymphoma, spinal axis tumors, brain stem glioma, pituitary adenoma, small cell lung; non-small lung, fallopian tubes, endometrium, colon, adenoma, thyroid, parathyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkins, hairy cells, buccal cavity and pharynx (oral), lip tongue, mouth, pharynx, small intestine, colon-rectum, large intestine, rectum, brain and central nervous system, and leukemia. The term encompasses tumor invasion, tumor growth, and/or tumor metastasis.

“Immunodeficiency diseases” includes diseases involving an immunodeficient state reflected in an inability of the immune system to respond to various antigens. An immunodeficient state may allow for the growth of tumors, due to a defect in the normal antitumor activity of the immune system or may lead to allergic or autoimmune problems. An immunodeficient state is often caused by metabolic disorders, for example, resulting from diabetes, obesity, artherosclerosis, uremia and attrition. AIDS or AIDS-related syndromes are examples of immunodeficiency diseases.

A “chemotherapeutic agent” refers to a chemical compound useful in the treatment of cancer. Examples of chemotherapeutic agents include Erlotinib (TARCEVA®, Genentech/OSI Pharm.), Bortezomib (VELCADE®, Millenium Pharm.), Fulvestrant (FASLODEX®, AstraZeneca), Sutent (SU11248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (GSK572016, Glaxo Smith Kline), Lonafarnib (SCH 66336), Sorafenib (BAY43-9006, Bayer Labs), Gefitinib (IRESSA®, AstraZeneca), AG1478, AG1571 (SU 5271; Sugen), alkylating agents such as thiotepa and CYTOXAN®, cyclosphosphamide, alkyl sulfonates such as busulfan, improsulfan and piposulfan, aziridines such as benzodopa, carboquone, meturedopa, and uredopa, ethylenimines and methylamelamines including altretamine, triethylenemelamine, triethylenephosphoramide, triethylenethiophosphoramide and trimethylomelamine, acetogenins, a camptothecin, bryostatin, callystatin, CC-1065, cryptophycins, dolastatin, duocarmycin, eleutherobin, pancratistatin, a sarcodictyin, spongistatin, nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard, nitrosureas such as carmustine, chlorozotocin, fotemustine, lomustine, nimustine, and ranimnustine, antibiotics such as the enediyne antibiotics, aclacinomysins, actinomycin, authramycin, azaserine, bleomycins, cactinomycin, carabicin, carminomycin, carzinophilin, chromomycinis, dactinomycin, daunorubicin, detorubicih, 6-diazo-5-oxo-L-norleucine, ADRIAMYCIN®, (doxorubicin, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin, anti-metabolites such as methotrexate and 5-fluorouracil (5-FU), folic acid analogues such as denopterin, methotrexate, pteropterin, trimetrexate, purine analogs such as fludarabine, 6-mercaptopurine, thiamiprine, thioguanine, pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elformithine, elliptinium acetate, an epothilone, etoglucid, gallium nitrate, hydroxyurea, lentinan, lonidainine, maytansinoids such as maytansine and ansamitocins, mitoguazone, mitoxantrone, mopidanmol, nitraerine, pentostatin, phenamet, pirarubicin, losoxantrone, podophyllinic acid, 2-ethylhydrazide, procarbazine, PSK® polysaccharide complex (JHS Natural Products, Eugene, Oreg.), razoxane, rhizoxin, sizofuran, spirogermanium, tenuazonic acid, triaziquone, 2,2′,2″-trichlorotriethylamine, trichothecenes (especially T-2 toxin, verracurin A, roridin A and anguidine), urethane, vindesine, dacarbazine, mannomustine, mitobronitol, mitolactol, pipobroman, gacytosine, arabinoside (“Ara-C”), cyclophosphamide, thiotepa, taxoids, e.g., TAXOL® (paclitaxel; Bristol-Myers Squibb Oncology, Princeton, N. J.), ABRAXANE® (Cremophor-free), albumin-engineered nanoparticle formulations of paclitaxel (American Pharmaceutical Partners, Schaumberg, Ill.), and TAXOTERE® (doxetaxel; Rhone-Poulenc Rorer, Antony, France), chloranbucil, GEMZAR® (gemcitabine), 6-thioguanine, mercaptopurine, methotrexate, platinum analogs such as cisplatin and carboplatin, vinblastine, etoposide (VP-16), ifosfamide, mitoxantrone, vincristine, NAVELBINE® (vinorelbine), novantrone, teniposide, edatrexate, daunomycin, aminopterin, xeloda, ibandronate, CPT-11, topoisomerase inhibitor RFS 2000, difluorometlhylomithine (DMFO), retinoids such as retinoic acid, capecitabine, and pharmaceutically acceptable salts, acids and derivatives of any of the above.

Compounds

The invention provides compounds of the formula I that enhance cytokine signalling, in particular enhance TGF-β dependent Smad2/3 nuclear translocation, wherein in the definition of the compounds * is ═C or ═O, R1 and R2 independently represent substituted or unsubstituted alkyl, alkenyl, alkynyl, alkylene, alkenylene, alkoxy, alkenyloxy, cycloalkyl, cycloalkenyl, aryl, aryloxy, arylalkoxy, aroyl, heteroaryl, heterocyclic, acyl, acyloxy, sulfonyl, sulfinyl, sulfenyl, amino, imino, azido, thiol, thioalkyl, thioalkoxy, thioaryl, nitro, ureido, cyano, halo, silyl, silyloxy, silythio, carboxyl, carbonyl, carbamoyl, carboxamide, carboxylic ester, phosphono, a substituted or unsubstituted aryl group fused to a cycloalkyl group, or a pharmaceutically acceptable salt thereof.

In classes of compounds of the formula I, R1 represents hydrogen, substituted or unsubstituted alkyl, alkenyl, alkynyl, alkylene, alkoxy, acyl, acyloxy, sulfonyl, sulfinyl, sulfenyl, amino, imino, azido, thiol, thioalkyl, thioalkoxy, nitro, cyano, halo, silyl, silyloxy, silythio, carboxyl, carbonyl, carbamoyl, or carboxamide. In particular classes of compounds of the formula I, R1 represents hydrogen, substituted or unsubstituted alkyl, alkenyl, alkoxy, amino, imino, azido, thiol, thioalkyl, nitro, cyano, halo, silyl, or carboxyl. In more particular classes of compounds of the formula I, R1 represents hydrogen or substituted or unsubstituted alkyl. In aspects of the invention, R1 represents C₁-C₃ alkyl substituted with one or more halo, in particular fluoro or chloro, more particularly fluoro. In an embodiment, R1 represents methyl substituted with one, two, or three halo, in particular fluoro.

In classes of compounds of the formula I, R2 represents —NR15 wherein R15 is —R16R17 wherein R16 is substituted or unsubstituted C₁-C₆ alkyl, and R17 is substituted or unsubstituted aryl. In particular classes of the compounds of the formula I, R2 represents —NR15 wherein R15 is —R16R17 wherein R16 is unsubstituted C₁-C₆ alkyl, and R17 is substituted or unsubstituted aryl. In more particular classes of the compounds of the formula I, R2 represents —NR15 wherein R15 is —R16R17 wherein R16 is C₁-C₆ alkyl, and R17 is substituted aryl. In other more particular classes of the compounds of the formula I, R2 represents —NR15 wherein R15 is —R16R17 wherein R16 is methyl, ethyl, or propyl, and R17 is substituted or unsubstituted phenyl. In embodiments of classes of the compounds of the formula I, R2 represents —NR15 wherein R15 is —R16R17 wherein R16 is ethyl or propyl, and R17 is phenyl substituted with alkoxy, in particular methoxy, ethoxy, or propoxy, more particularly methoxy.

In classes of compounds of the formula I, R2 represents —NR15 wherein R15 is an substituted or unsubstituted heterocylic with 1 to 2 oxygen atoms and 1 to 3 nitrogen atoms. In particular classes of compounds of the formula I, R2 represents —NR15 wherein R15 is an substituted or unsubstituted heterocylic with 1 oxygen atom and 2 nitrogen atoms. In more particular classes of compounds of the formula I, R2 represents —NR15 wherein R15 is an substituted or unsubstituted 1,3,4-oxadiazolyl. In aspects of the invention, R15 is substituted with hydroxyl, alkyl, halo, alkyl, alkenyl, alkynyl, alkylene, alkenylene, alkoxy, alkenyloxy, cycloalkyl, cycloalkenyl, aryl, aryloxy, arylalkoxy, aroyl, heteroaryl, heterocyclic, acyl, acyloxy, sulfonyl, sulfinyl, sulfenyl, amino, imino, azido, thiol, thioalkyl, thioalkoxy, thioaryl, nitro, ureido, cyano, silyl, silyloxy, silythio, carboxyl, carbonyl, carbamoyl, carboxamide. In a particular aspect, R15 is aryl substituted with halo or substituted or unsubstituted alkyl. In more particular aspects R15 is phenyl substituted with bromo, chloro, or substituted C₁-C₃-alkyl (e.g., —(CH₃)₃).

In classes of compounds of the formula I, R2 represents a substituted or unsubstituted aryl fused to a cycloalkyl. In particular classes of compounds of the formula I, R2 represents substituted or unsubstituted phenyl fused with a cycloalkyl having 3 to 6 carbon atoms. In more particular classes of compounds of the formula I, R2 represents substituted or unsubstituted phenyl fused with a cyclopropyl, cyclobutyl or cyclopentyl, most particularly cyclopentyl. In aspects, R2 is phenyl fused to cyclopentyl wherein the phenyl is substituted with halo, alkyl, or oxy, more particularly ═O.

In classes of compounds of the formula I, * is ═C, R1 represents hydrogen, substituted or unsubstituted alkyl, alkenyl, alkynyl, alkylene, alkoxy, acyl, acyloxy, sulfonyl, sulfinyl, sulfenyl, amino, imino, azido, thiol, thioalkyl, thioalkoxy, nitro, cyano, halo, silyl, silyloxy, silythio, carboxyl, carbonyl, carbamoyl, or carboxamide, and R2 represents —NR15 wherein R15 is —R16R17 wherein R16 is C₁-C₆ alkyl, and R17 is substituted or unsubstituted aryl. In aspects, R1 is substituted C₁-C₆ alkyl and R2 is NR15 wherein R15 is —R16R17 wherein R16 is C₁-C₆ alkyl and R17 is substituted or unsubstituted aryl. In aspects, R1 is substituted C₁-C₃ alkyl and R2 is NR15 wherein R15 is —R16R17 wherein R16 is C₁-C₃ alkyl and R16 is substituted or unsubstituted aryl. In other aspects of the compounds of the formula I, R1 is C₁-₃ C₃ alkyl substituted with halo and R2 is NR15 wherein R15 is —R16R17 wherein R16 is C₁-C₃ alkyl and R16 is aryl. In particular aspects of the compounds of the formula I, R1 is C₁-C₃ alkyl substituted with halo and R2 is NR15 wherein R15 is —R16R17 wherein R16 is C₁-C₃ alkyl and R16 is substituted aryl. In further particular aspects, R1 is methyl substituted with one, two or three fluoro and R2 is NR15 wherein R15 is —R16R17 wherein R16 is C₁-C₃ alkyl and R16 is aryl which may be substituted with hydroxyl, halo, alkyl, alkenyl, alkynyl, alkylene, alkoxy, acyl, acyloxy, sulfonyl, sulfinyl, sulfenyl, amino, imino, azido, thiol, thioalkyl, thioalkoxy, nitro, cyano, halo, silyl, silyloxy, silythio, carboxyl, carbonyl, carbamoyl, or carboxamide. In still further particular aspects, R1 is methyl substituted with one, two or three fluoro and R2 is NR15 wherein R15 is —R16R17 wherein R16 is C₁-C₃ alkyl and R16 is aryl which may be substituted with hydroxyl, halo, or alkoxy, preferably alkoxy.

In classes of compounds of the formula I, * is ═C, R1 represents hydrogen, substituted or unsubstituted alkyl, alkenyl, alkynyl, alkylene, alkoxy, acyl, acyloxy, sulfonyl, sulfinyl, sulfenyl, amino, imino, azido, thiol, thioalkyl, thioalkoxy, nitro, cyano, halo, silyl, silyloxy, silythio, carboxyl, carbonyl, carbamoyl, or carboxamide, and R2 represents —NR15 wherein R15 is an optionally substituted heterocylic with 1 to 2 oxygen atoms and 1 to 3 nitrogen atoms.

In classes of compounds of the formula I, * is ═C, R1 represents hydrogen, substituted or unsubstituted alkyl, alkenyl, alkoxy, amino, imino, azido, thiol, thioalkyl, nitro, cyano, halo, silyl, or carboxyl and R2 represents —NR15 wherein R15 is an optionally substituted heterocylic with 1 to 2 oxygen atoms and 1 to 3 nitrogen atoms.

In classes of compounds of the formula I, * is ═C, R1 represents hydrogen or substituted or unsubstituted alkyl and R2 represents —NR15 wherein R15 is an optionally substituted heterocylic with 1 oxygen atom and 2 nitrogen atoms.

In classes of compounds of the formula I, * is ═C, R1 represents hydrogen or substituted or unsubstituted alkyl and R2 represents —NR15 wherein R15 is substituted or unsubstituted 1,3,4-oxadiazolyl. In classes of compounds of the formula I, * is ═C, R1 represents hydrogen or substituted or unsubstituted alkyl and R2 represents —NR15 wherein R15 is an 1,3,4-oxadiazole substituted with optionally substituted alkyl, alkenyl, alkynyl, alkylene, alkoxy, acyl, acyloxy, sulfonyl, sulfinyl, sulfenyl, amino, imino, azido, thiol, thioalkyl, thioalkoxy, nitro, cyano, halo, silyl, silyloxy, silythio, carboxyl, carbonyl, carbamoyl, or carboxamide, more particularly substituted alkyl or halo, most particularly —(CH₃)₃, chloro, or bromo.

In classes of compounds of the formula I, R2 represents —NR15 wherein R15 is —R16R17 wherein R16 is C₁-C₆ alkyl, and R17 is aryl, or R15 is an optionally substituted heterocylic with 1 to 2 oxygen atoms and 1 to 3 nitrogen atoms.

In classes of compounds of the formula I, * is ═O, R1 represents hydrogen, substituted or unsubstituted alkyl, alkenyl, alkynyl, alkylene, alkoxy, acyl, acyloxy, sulfonyl, sulfinyl, sulfenyl, amino, imino, azido, thiol, thioalkyl, thioalkoxy, nitro, cyano, halo, silyl, silyloxy, silythio, carboxyl, carbonyl, carbamoyl, or carboxamide, and R2 is a substituted or unsubstituted aryl fused to a cycloalkyl. In particular classes of compounds of the formula I, * is ═O, R1 represents hydrogen, substituted or unsubstituted alkyl or alkoxy, sulfonyl, sulfinyl, sulfenyl, amino, imino, azido, thiol, nitro, cyano, halo, silyl, carboxyl, carbonyl, or carbamoyl, and R2 is a substituted or unsubstituted aryl fused to a cycloalkyl having 3 to 6 carbon atoms. In more particular classes of compounds of the formula I, * is ═O, R1 represents hydrogen or substituted or unsubstituted alkyl and R2 is substituted or unsubstituted phenyl fused to a cycloalkyl having 3 to 6 carbon atoms, more particularly cyclopropyl, cyclobutyl or cyclopentyl. In an aspect, R2 is substituted phenyl fused to cyclopropyl. In particular embodiments, R2 is phenyl substituted with halo, ═O, alkyl, or alkoxy, fused to cyclopropyl at positions 2 to 3.

In aspects of the invention, a compound of the formula I excludes compounds of the formula I wherein when (i) R1 is —NH—CH₂CH₂-Ph, R2 is —CF₃, and/or, (ii) R1 is an oxadiazolyl substituted with chlorophenyl, when R2 is hydrogen.

In embodiments of the invention, a compound of the formula I is a compound listed in Table 1, in particular having a structure of a diethyl 2 (anilinomethylene) malonate compound or derivative thereof. In particular embodiments, a compound of the formula I is the compound identified in Table I as DAM-1976

Therapeutic efficacy and toxicity of compounds, compositions and methods of the invention may be determined by standard pharmaceutical procedures in cell cultures or with experimental animals such as by calculating a statistical parameter such as the ED₅₀ (the dose that is therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Pharmaceutical compositions which exhibit large therapeutic indices are preferred.

Process

The compounds of the formula I of this invention may be prepared using reactions and methods generally known to the person of ordinary skill in the art, having regard to that knowledge and the disclosure of this application including the Examples. The reactions are performed in a solvent appropriate to the reagents and materials used and suitable for the reactions being effected. It will be understood by those skilled in the art of organic synthesis that the functionality present on the compounds should be consistent with the proposed reaction steps. This will sometimes require modification of the order of the synthetic steps or selection of one particular process scheme over another in order to obtain a desired compound of the invention. It will also be recognized that another major consideration in the development of a synthetic route is the selection of the protecting group used for protection of the reactive functional groups present in the compounds described in this invention. An authoritative account describing the many alternatives to the skilled artisan is Greene and Wuts (Protective Groups In Organic Synthesis, Wiley and Sons, 1991).

The starting materials and reagents used in preparing compounds of the invention are either available from commercial suppliers such as the Aldrich Chemical Company (Milwaukee, Wis.), Bachem (Torrance, Calif.), Sigma (St. Louis, Mo.), or Lancaster Synthesis Inc. (Windham, N.H.) or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

Generally, a compound of the formula I may be prepared by a condensation reaction between diethylmalonate (CAS Number 105-53-3) and the appropriate aldehyde in a Knoevenagel reaction, for example, according to the following scheme:

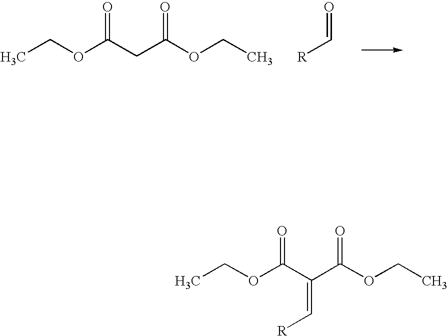

The starting materials, intermediates, and compounds of this invention may be isolated and purified using conventional techniques, such as precipitation, filtration, distillation, crystallization, chromatography, and the like. The compounds may be characterized using conventional methods, including physical constants and spectroscopic methods, in particular HPLC.

The compounds of the formula I which are basic in nature can form a wide variety of different salts with various inorganic and organic acids. In practice it is desirable to first isolate a compound of the formula I from the reaction mixture as a pharmaceutically unacceptable salt and then convert the latter to the free base compound by treatment with an alkaline reagent and subsequently convert the free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent such as methanol or ethanol. Upon careful evaporation of the solvent, the desired solid salt is obtained.

Compounds of the formula I which are acidic in nature are capable of forming base salts with various pharmacologically acceptable cations. These salts may be prepared by conventional techniques by treating the corresponding acidic compounds with an aqueous solution containing the desired pharmacologically acceptable cations and then evaporating the resulting solution to dryness, preferably under reduced pressure. Alternatively, they may be prepared by mixing lower alkanolic solutions of the acidic compounds and the desired alkali metal alkoxide together and then evaporating the resulting solution to dryness in the same manner as before. In either case, stoichiometric quantities of reagents are typically employed to ensure completeness of reaction and maximum product yields.

Compositions and Kits

A compound of the Formula I may be formulated into a pharmaceutical composition for administration to a subject. Pharmaceutical compositions comprising a compound of the formula I generally further comprise suitable pharmaceutically acceptable carriers, excipients, and vehicles selected based on the intended form of administration, and consistent with conventional pharmaceutical practices. Suitable pharmaceutical carriers, excipients, and vehicles are described in the standard text, in Remington. The Science and Practice of Pharmacy (21^{st} Edition. 2005, University of the Sciences in Philadelphia (Editor), Mack Publishing Company), and in The United States Pharmacopeia: The National Formulary (USP 24 NF 19) published in 1999.

By way of example, for oral administration in the form of a capsule or tablet, the active components can be combined with an oral, non-toxic pharmaceutically acceptable inert carrier such as lactose, starch, sucrose, methyl cellulose, magnesium stearate, glucose, calcium sulfate, dicalcium phosphate, mannitol, sorbital, and the like. For oral administration in a liquid form, the drug components may be combined with any oral, non-toxic, pharmaceutically acceptable inert carrier such as ethanol, glycerol, water, and the like. Suitable binders (e.g. gelatin, starch, corn sweeteners, natural sugars including glucose; natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, and coloring agents may also be combined in the compositions or components thereof. Compositions as described herein can further comprise wetting or emulsifying agents, or pH buffering agents.

A composition of the invention can be a liquid solution, suspension, emulsion, tablet, pill, capsule, sustained release formulation, or powder. The compositions can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, etc. Various delivery systems are known and can be used to administer a composition of the invention, e.g. encapsulation in liposomes, microparticles, microcapsules, and the like.

Formulations for parenteral administration may include aqueous solutions, syrups, aqueous or oil suspensions and emulsions with edible oil such as cottonseed oil, coconut oil or peanut oil. Dispersing or suspending agents that can be used for aqueous suspensions include synthetic or natural gums, such as tragacanth, alginate, acacia, dextran, sodium carboxymethylcellulose, gelatin, methylcellulose, and polyvinylpyrrolidone.

Compositions for parenteral administration may include sterile aqueous or non-aqueous solvents, such as water, isotonic saline, isotonic glucose solution, buffer solution, or other solvents conveniently used for parenteral administration of therapeutically active agents. A composition intended for parenteral administration may also include conventional additives such as stabilizers, buffers, or preservatives, e.g. antioxidants such as methylhydroxybenzoate or similar additives.

Compositions of the invention can be formulated as pharmaceutically acceptable salts as described herein.

A composition of the invention may be sterilized by, for example, filtration through a bacteria retaining filter, addition of sterilizing agents to the composition, irradiation of the composition, or heating the composition. Alternatively, the compounds or compositions of the present invention may be provided as sterile solid preparations e.g. lyophilized powder, which are readily dissolved in sterile solvent immediately prior to use.

After pharmaceutical compositions have been prepared, they can be placed in an appropriate container and labeled for treatment of an indicated condition. For administration of a composition of the invention, such labeling would include amount, frequency, and method of administration.

The invention also provides a kit comprising a compound or a pharmaceutical composition of the invention. The kit can be a package which houses a container which contains a composition of the invention and also houses instructions for administering the composition to a subject. In an aspect, the invention provides a pharmaceutical pack or kit comprising one or more containers filled with a compound of the invention or one or more of the ingredients of a pharmaceutical composition of the invention to provide a beneficial effect, in particular a sustained beneficial effect. Associated with such container(s) can be various written materials such as instructions for use, or a notice in the form prescribed by a governmental agency regulating the labeling, manufacture, use or sale of pharmaceuticals or biological products, which notice reflects approval by the agency of manufacture, use, or sale for human administration.

Preferred compositions and kits of the invention comprise compounds with a core chemical structure of diethyl 2-(anilinomethylene)malonate (DAM), more preferably compounds of the Formula I depicted in Table 1, and most preferably DAM- 1976.

Applications

The invention contemplates the use of compounds of the Formula I and compositions comprising the same for treating a disease or disorder disclosed herein, in particular preventing, and/or ameliorating disease severity, disease symptoms, and/or periodicity of recurrence of a disease disclosed herein. The invention also contemplates treating disorders disclosed herein in mammals using the compounds, compositions or treatments of the invention.

With reference to the following examples and related discussions, the present invention provides various methods relating to enhancing sensitivity to cytokines, enhancing/restoring responsiveness to EGF and TGFP, slowing cell proliferation, slowing trafficking of receptors, integrins, and transporters that contribute to cellular phenotypes, stabilizing microfilaments, slowing endocytosis, and/or delaying cell cycle progression. As illustrated more fully elsewhere herein, such methods include but are not limited to use of the compounds and compositions of this invention, preferably in a dose dependent fashion, to selectively slow or reduce tumor cell growth. Such methods can include the preparation and/or formulation of a composition with subsequent administration and/or delivery to cells, tissue, culture or a related physiological system or medium, such administration/delivery in a dose or at a compositional concentration sufficient to effect the desired regulation and/or inhibition, without substantially inhibiting other desired endogenous cytokine responses.

In an aspect, the present invention relates to the enhancement of sensitivity to cytokines by administering a therapeutically effective amount of a compound of the formula I or a composition comprising a compound of the formula I.

In an aspect, the present invention provides methods of treating or preventing proliferative diseases by enhancing sensitivity to cytokines comprising administering a compound of the Formula I, a pharmaceutically acceptable salt thereof, or a composition comprising a compound of the formula I, and a pharmaceutically acceptable carrier, excipient, or vehicle.

In embodiments, the present invention provides methods of treating or preventing proliferative diseases by enhancing sensitivity to cytokines comprising administering compositions comprising one or more of the compounds of the Formula I, in particular one or more compounds depicted in Table 1, or derivatives of these compounds.

In other embodiments, the invention provides a method for reducing or inhibiting tumor metastasis, comprising administering to a subject in need thereof a therapeutically effective amount of a compound of the Formula I, a pharmaceutically acceptable salt thereof, or a composition comprising a compound of the formula I, and a pharmaceutically acceptable carrier, excipient, or vehicle.

The present invention also provides compounds (e.g., compounds listed in Table 1) for use. in enhancing cytokine signaling; enhancing TGF-β dependent Smad2/3 nuclear translocation; targeting cells with a D₅₀ value of about 1-4 μM; inhibiting microfilament remodelling; enhancing sensitivity to cytokines by prolonging the trafficking and activation of signaling intermediates; stabilizing microfilaments; slowing endocytosis of EGFR, increasing sensitivity to EGF and TGF-β cytokines; increasing basal nuclear Erk-p and Smad2/3; suppressing tumor cell growth; and/or, modulating receptor trafficking both before and after ligand binding.

The invention further provides a method involving administering to a subject a therapeutic compound of the formula I, or a pharmaceutically acceptable salt thereof, or a composition comprising a compound of the formula I, and a pharmaceutically acceptable carrier, excipient, or vehicle, that enhance cytokine signaling; enhance TGF-β dependent Smad2/3 nuclear translocation; target cells with a D₅₀ value of about 1-4 μM; inhibit microfilament remodelling; enhance sensitivity to cytokines by prolonging the trafficking and activation of signaling intermediates; stabilize microfilaments; slow endocytosis of EGFR, increase sensitivity to EGF and TGF-β cytokines; increase basal nuclear Erk-p and Smad2/3; suppress tumor cell growth; and/or, modulate receptor trafficking both before and after ligand binding.

The invention further provides a method involving administering to a subject a therapeutic compound of the formula I, or a pharmaceutically acceptable salt thereof, or a composition comprising a compound of the formula I, and a pharmaceutically acceptable carrier, excipient, or vehicle, that enhance cytokine signaling; enhance TGF-β dependent Smad2/3 nuclear translocation; target cells with a D₅₀ value of about 1-4 μM; inhibit microfilament remodelling; enhance sensitivity to cytokines by prolonging the trafficking and activation of signaling intermediates; stabilize microfilaments; slow endocytosis of EGFR, increase sensitivity to EGF and TGF-β cytokines; increase basal nuclear Erk-p and Smad2/3; suppress tumor cell growth; and/or, modulate receptor trafficking both before and after ligand binding.

The invention provides a method involving administering to a subject a therapeutically effective amount of a compound of the formula I, or a pharmaceutically acceptable salt thereof, or a composition comprising a compound of the formula I, and a pharmaceutically acceptable carrier, excipient, or vehicle.

In aspects of the invention, the compounds or compositions of the invention are provided to an individual displaying characteristics of a proliferative disease (e.g., cancer), such that treatment with the compounds results in modulation of such characteristics, for example, a decrease of reduced sensitivity to EGF and TGF-β.

In an aspect of the invention, a method is provided for treating in a subject a disease involving or characterized by abnormal cytokine signaling, in particular abnormal EGF and TGF-β cytokines, comprising administering to the subject a therapeutically effective amount of a compound of the Formula I, a pharmaceutically acceptable salt thereof, or a composition comprising a compound of the formula I, and a pharmaceutically acceptable carrier, excipient, or vehicle.

In another aspect, a method is provided for treating in a subject a condition involving abnormal cell growth, comprising administering to the subject a therapeutically effective amount of a composition comprising a compound of the Formula I, a pharmaceutically acceptable salt thereof, or a composition comprising a compound of the formula I, and a pharmaceutically acceptable carrier, excipient, or vehicle.

In another aspect, the invention provides a method for treating in a subject a disease associated with abnormal cell growth that can be decreased or inhibited with a compound disclosed herein comprising administering to the subject a therapeutically effective amount of a compound of the formula I, a pharmaceutically acceptable salt thereof, or a composition comprising a compound of the formula I and a pharmaceutically acceptable carrier, excipient, or vehicle.

In a further aspect, the invention provides a method for reducing or inhibiting abnormal cell growth in a subject comprising administering to the subject a therapeutically effective amount of a compound of the formula I, a pharmaceutically acceptable salt thereof, or a composition comprising a compound of the formula I and a pharmaceutically acceptable carrier, excipient, or vehicle.

In some embodiments, the invention provides methods of enhancing cell signaling molecule production (e.g., TGFP and/or EGF), comprising administering a compound of the formula I, a pharmaceutically acceptable salt thereof, or a composition comprising a compound of the formula I and a pharmaceutically acceptable carrier, excipient, or vehicle.

In particular embodiments, the invention provides methods of enhancing cell signaling molecule production (e.g., TGFβ and/or EGF), comprising administering compositions comprising one or more compounds of the Formula I, in particular the compounds depicted in Table 1, or derivatives of these compounds.

In an aspect, the invention provides a method for amelioriating progression of a disorder or disease or obtaining a less severe stage of a disorder or disease disclosed herein in a subject suffering from such disorder or disease comprising administering a therapeutically effective amount of a compound of the Formula I, a pharmaceutically acceptable salt thereof, or a composition comprising a compound of the Formula I and a pharmaceutically acceptable carrier, excipient, or vehicle.

The invention relates to a method of delaying the progression of a disorder disclosed herein comprising administering a therapeutically effective amount of a compound of the Formula I, a pharmaceutically acceptable salt thereof, or a composition comprising a compound of the Formula I and a pharmaceutically acceptable carrier, excipient, or vehicle.

The invention also relates to a method of increasing survival of a subject suffering from a disorder disclosed herein comprising administering a therapeutically effective amount of a compound of the Formula I, a pharmaceutically acceptable salt thereof, or a composition comprising a compound of the Formula I and a pharmaceutically acceptable carrier, excipient, or vehicle.

The invention has particular applications in treating or preventing a proliferative disease, in particular cancer. In an aspect, the invention provides a method for treating or inhibiting a proliferative disease in a patient in need thereof which includes administering to the individual a composition that provides a compound of the formula I in a dose sufficient to enhance sensitivity to cytokine signalling, in particular for a prolonged period following administration.

In an aspect of the invention a compound of the Formula I is utilized in the treatment or inhibition of a cancer. A cancer may be treated or inhibited by administering a therapeutically effective amount of a compound of the Formula I, a pharmaceutically acceptable salt thereof, or a composition comprising a compound of the Formula I and a pharmaceutically acceptable carrier, excipient, or vehicle. Such treatment may be effective for retarding the effects of a cancer.

In another aspect, the invention provides a method for treating or inhibiting a cancer by providing a composition comprising a compound of the invention in an amount sufficient to enhance sensitivity to cytokine signalling, in particular for a prolonged period following administration.

In as aspect, the invention relates to a method of treatment comprising administering a therapeutically effective amount of one or more compound of the Formula I, a pharmaceutically acceptable salt thereof, or a composition comprising a compound of the Formula I and a pharmaceutically acceptable carrier, excipient, or vehicle, which upon administration to a subject with symptoms of a proliferative disease, produces one or more therapeutic effect, in particular a beneficial effect, more particularly a sustained beneficial effect. In an embodiment, a beneficial effect is evidenced by reduction or inhibition of tumor growth, invasion, and/or metastasis, increased lifespan, and/or increased survival.

Compounds, pharmaceutical compositions and methods of the invention can be selected that have sustained beneficial effects. In an embodiment, a pharmaceutical composition with statistically significant sustained beneficial effects is provided comprising a therapeutically effective amount of a compound of the invention.

The invention provides a method of preventing a disorder disclosed herein in a subject with a genetic predisposition to such disorder by administering an effective amount of a compound of the Formula I, or a pharmaceutically acceptable salt thereof, or a composition comprising a compound of the Formula I and a pharmaceutically acceptable carrier, excipient, or vehicle.

In an aspect, the invention relates to a method of improving the lifespan of a subject suffering from a disorder or disease disclosed herein, in particular a proliferative disease, comprising administering a therapeutically effective amount of a compound of the Formula I, a pharmaceutically acceptable salt thereof, or a composition comprising a compound of the Formula I and a pharmaceutically acceptable carrier, excipient, or vehicle.

In some aspects, greater efficacy and potency of a treatment of the invention may improve the therapeutic ratio of treatment, reducing untoward side effects and toxicity. Selected methods of the invention may also improve long-standing disease even when treatment is begun long after the appearance of symptoms.

The compositions and methods described herein are indicated as therapeutic agents or methods either alone or in conjunction with other therapeutic agents or other forms of treatment. They may be combined or formulated with one or more therapies or agents used to treat a condition described herein. Compositions of the invention may be administered concurrently, separately, or sequentially with other therapeutic agents or therapies. Therefore, the compounds of the Formula I may be co-administered with one or more additional therapeutic agents, and agents that are used for the treatment of complications resulting from or associated with a disorder, or general medications that treat or prevent side effects. Examples of additional therapeutic agents include without limitation antitumor agents, alkylating agents, antimetabolites, antibiotics, plant-derived antitumor agents, campothecin derivatives, tyrosine kinase inhibitors, antibodies, interferons and/or biological modifiers. In aspects of the invention for treating cancer, a chemotherapeutic agent may additionally be administered.

In some aspects, one or more of the compounds of the invention, in particular the compounds depicted in Table 1, or derivatives of these compounds, are co-administered with other recognized therapeutics to treat proliferative diseases In some embodiments, the compounds are provided to an individual displaying characteristics of a proliferative disease (e.g., cancer), such that treatment with the compounds enhances cytokine signaling production which is reduced or inhibited as a result of the disease.

The invention also contemplates the use of a composition comprising at least one compound of the formula I for the preparation of a medicament in treating a disorder disclosed herein (e.g. a proliferative disease). In an embodiment, the invention relates to the use of a therapeutically effective amount of at least one compound of the invention for preparation of a medicament for providing therapeutic effects, in particular beneficial effects, more particularly sustained beneficial effects, in treating a disorder disclosed herein. In a still further embodiment the invention provides the use of a compound of the invention for the preparation of a medicament for prolonged or sustained treatment of a disorder disclosed herein.

Preferred methods and uses of the invention comprise compounds with a core chemical structure of diethyl 2-(anilinomethylene)malonate (DAM), more preferably compounds of the Formula I depicted in Table 1, and most preferably DAM-1976.

Administration

Compounds and compositions of the present invention can be administered by any means that produce contact of the active agent(s) with the agent's sites of action in the body of a subject or patient to produce a therapeutic effect, in particular a beneficial effect, in particular a sustained beneficial effect. The active ingredients can be administered simultaneously or sequentially and in any order at different points in time to provide the desired beneficial effects. A compound and composition of the invention can be formulated for sustained release, for delivery locally or systemically. It lies within the capability of a skilled physician or veterinarian to select a form and route of administration that optimizes the effects of the compositions and treatments of the present invention to provide therapeutic effects, in particular beneficial effects, more particularly sustained beneficial effects.

The compositions may be administered in oral dosage forms such as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular forms, all utilizing dosage forms well known to those of ordinary skill in the pharmaceutical arts. The compositions of the invention may be administered by intranasal route via topical use of suitable intranasal vehicles, or via a transdermal route, for example using conventional transdermal skin patches. A dosage protocol for administration using a transdermal delivery system may be continuous rather than intermittent throughout the dosage regimen. A sustained release formulation can also be used for the therapeutic agents.

An amount of a therapeutic of the invention which will be effective in the treatment of a particular disorder disclosed herein to provide effects, in particular beneficial effects, more particularly sustained beneficial effects, will depend on the nature of the disorder, and can be determined by standard clinical techniques. The precise dose to be employed in the formulation will also depend on the route of administration, and the seriousness of the disorder, and should be decided according to the judgment of the practitioner and each patient's circumstances.

Thus, the dosage regimen of the invention will vary depending upon known factors such as the pharmacodynamic characteristics of the agents and their mode and route of administration; the species, age, sex, health, medical condition, and weight of the patient, the nature and extent of the symptoms, the kind of concurrent treatment, the frequency of treatment, the route of administration, the renal and hepatic function of the patient, and the desired effect.

Suitable dosage ranges for administration are particularly selected to provide therapeutic effects, in particular beneficial effects, more particularly sustained beneficial effects. A dosage range is generally effective for triggering the desired biological responses. The dosage ranges are generally about 0.5 mg to about 2 g per kg, about 1 mg to about 1 g per kg, about 1 mg to about 500 mg per kg, about 1 mg to about 400 mg per kg, about 1 mg to about 300 mg per kg, about 1 mg to about 200 mg per kg, about 1 mg to about 100 mg per kg, about 1 mg to about 50 mg per kg, or about 10 mg to about 100 mg per kg of the weight of a subject.

A composition or treatment of the invention may comprise a unit dosage of at least one compound of the invention to provide beneficial effects. A “unit dosage” or “dosage unit” refers to a unitary i.e. a single dose which is capable of being administered to a patient, and which may be readily handled and packed, remaining as a physically and chemically stable unit dose comprising either the active agents as such or a mixture with one or more solid or liquid pharmaceutical excipients, carriers, or vehicles.

A subject may be treated with a compound of the Formula I or composition or formulation thereof on substantially any desired schedule. A composition of the invention may be administered one or more times per day, in particular 1 or 2 times per day, once per week, once a month or continuously. However, a subject may be treated less frequently, such as every other day or once a week, or more frequently. A compound, composition or formulation of the invention may be administered to a subject for about or at least about 1 week, 2 weeks to 4 weeks, 2 weeks to 6 weeks, 2 weeks to 8 weeks, 2 weeks to 10 weeks, 2 weeks to 12 weeks, 2 weeks to 14 weeks, 2 weeks to 16 weeks, 2 weeks to 6 months, 2 weeks to 12 months, 2 weeks to 18 months, or 2 weeks to 24 months, periodically or continuously.

The following example further illustrates the invention. The example is offered for illustrative purposes, and is not intended to limit the invention in any manner.

### EXAMPLE

The Following Materials and Methods were Used in the Study Described in this Example.

Cell Lines

Murine NMuMG epithelial cells were purchased from American Type Culture Collection and maintained in DMEM supplemented with 10% FBS and 10 μg/ml insulin (Gibco, Carlsbad, Calif.). Mammary tumor cell lines were established from spontaneous mammary carcinomas in MMTV-PyMT transgenic mice on a 129sv×FVB background with either Mgat5^{+/+} or Mgat5^{-/-} genotypes as previously described (18). The cell lines Mgat5^{+/+}(2.6) and Mgat5^{-/-}(22.9) were generated from mammary tumors removed from littermate PyMT transgenic mice (15).

Nuclear Translocation Assay

NMuMG epithelial cells were plated in 96 well Coming tissue plates (Coming) at a density of 5000 cells/well in 100 μl of DMEM, 10% FBS, 10 μg/ml insulin (Gibco) for 24 h. The medium was replaced with 100 μl of DMEM, 10 μg/ml insulin, 0.2 mg/ml BSA (fraction V) for 18 h. Test compounds at 10 μM were added and cells were incubated for 60 min at 37° C. TGF-β1 (R&D System, Minneapolis, Minn.) was added at 50 pM and incubated for a further 50 min. Cells were then fixed with 4% paraformaldehyde in PBS for 15 min and washed 3 times in PBS. Cells were permeabilized with 100% methanol for 2 min, then washed 3 times with PBS, followed by the addition of 150 μl PBS, 10% FBS. Plates were stored overnight at 4° C. Anti-Smad2/3 or anti-phospho-Smad2 antibodies (Transduction Laboratories, San Jose, Calif.) diluted 1:1000 in PBS plus 10% FBS were added at 50 μl/well and the plates were incubated for 1 h at 20° C. After 3 washes in PBS, Alexa Fluor 488 anti-mouse (Molecular Probes, Eugene, Oreg.) 1:1000 in PBS plus 10% FBS, plus 1:2000 Hoechst stain (Molecular Probes) was added for 1 h at 20° C. The wells were washed 3 times with PBS and imaged and quantified on the Array Scan (Cellomics Inc., Pittsburgh, Pa.) using the cytoplasmic-nuclear translocation program. To measure Erk phosphorylation (Erk-p) and nuclear translocation, mouse anti-phospho-Erkl/2 (Thr202/Tyr204) (Sigma, St. Louis, Mo.) was added at 1/1000 in PBS plus 10% FBS for 1 h at 20° C. The nuclear-cytoplasmic difference was determined for 100 cells per well, generating a mean±SE for each condition.

High Throughput Cell-based Screening Assay

For assay constancy, aliquots of NMuMG cells were stored in liquid nitrogen and expanded for 5-7 days immediately before their use in the screen. Cells were plated in 96 well plates manually, and cultured for 24 h in DMEM plus 10% FBS. After a further 18 h of serum starvation, the plates were placed on a fully automated system based on a ThermoCRS A255 robotic arm running on a 3-meter rail. A Multimek96 outfitted with FPS100H slotted pins (V&P Scientific, Sunnyvale Calif.) was used to transfer 200 nl of 5 mM compound stocks dissolved in DMSO to the 50 μl of cell culture. After 1 hr incubation with individual compounds at 10 μM, TGF-β1 (50 pM final concentration) was added using a fixed tip multiprobe HT II. The 50K compound library used in the screen was the Maybridge Diversity (Fisher) library. After incubating for 45 min with cytokine at 37° C., cells were fixed by the addition of 100 μl of 8% paraformaldehyde in PBS directly to the culture using a Multidrop 384. After 10 min, the cells were washed 3 times with PBS (no Ca⁺⁺, no Mg⁺⁺) using a Biotek Elx405 Magna washer and immediately permeabilized for 2 min±5 s at 20° C. by adding 100 μl of 100% methanol using the Multidrop 384, then washed 3 times with PBS in the Biotek Elx405 Magna washer, and incubated with PBS plus 10% FBS for 1 h at 20° C. Plates were then stored at 4° C. until the start of the staining procedure 16 h later, and this fully automated system processed up to 28 plates per 8 h shift. The automated staining of the cells was performed as described for the manual method using the Biotek ELx405 washer and Multiprobe HTII. Following antibody staining, the plates were stored at 4° C. in the dark for up to 48 h until they could be read on the Cellomics Array Scan. The assay development procedure required careful attention to the stability of reagents, as well as each of the 15 operations on the robotic platform. The Z′ factor is defined as the ratio of separation band to dynamic range of the assay based on positive and negative control data in the assay. It takes the formula Z′=1−[3σ_{c+}+3σ_{c-}]/[μ_{c+}−μ_{c-}] (19). The overall Z′ factor for the screen was 0.63.

Immunoblot Analysis

Cells were lysed in TNTE [50 mM Tris-HCl pH7.4, 150 mM NaCl, 1% Triton X-100, 1 mM EDTA, protease Inhibitor cocktail (Sigma)] solution. Western blots were performed with antibodies to phosphorylated Erk1/2 kinase (Thr202/Tyr204) (Sigma) or to phosphotyrosine (4G10, Upstate, Chicago, Ill.).

Immunofluoresent Microscopy

To monitor the adhesion junctions and actin microfilament, cells were grown in DMEM, 10% FBS on glass coverslips for 24h and then treated with DAM-01976 for 24-48 h. Cells were fixed with 3.7% formaldehyde for 15 min at 20° C. and permeabilized with 0.2% Triton-X100 for 5 min at 20° C. Cells were blocked with PBS-10% FBS for overnight at 4° C., incubated with 1:200 dilution of anti-E-cadherin antibody (BD Biosciences, San Jose, Calif.) for 2 h at 20° C. After 3 washes with PBS, cells were incubated with TRITC-phalloidin diluted 1:400 (Sigma) and a 1:2000 dilution of Hoechst (Sigma).

Cytotoxicity Assay

Viable cell counts were measured using the XTT-based Cell Proliferation Kit IIa (Roche, Mannheim, Germany), or with AlamarBlue (Sigma), following the suggested protocols. In brief, 1000 cells/well were seeded in 96 well plates and cultured for 24 h prior to the addition of DAM-1976. Viable cells were measured at 48 or 72 h by adding of either XTT or AlamarBlue with further incubation at 37° C. For the XTT assay, absorbance was measured at 500 nm, 4 h after addition of XTT. Fluorescence with excitation at 544 nm and emission at 590 nm were measured 24 h after addition of AlamarBlue.

Cell Spreading

Cells were plated at 1000 cells/well onto 96 well plates coated with 0.5 μg/well of fibronecinm (Sigma). After 4 h incubation at 37° C. in serum-free DMEM, cells were fixed and stained with TRITC-phalloidin. Cell area was quantified by Scan Array and expressed as the mean±SE of 100 cells/well. To measure microfilament turnover, cells growing in DMEM plus 10% FBS were treated with 100 ng/ml Latrunculin-A, an actin monomer-binding drug that renders the monomers incompetent for filament formation. At times after treatment the cell area and microfilament density was measured following staining with TRITC-phalloidin.

Distribution of Cytokine Receptors

To measure surface EGFR, proteins were biotinylated by incubation with 0.5 mg/ml sulfosuccinimidyl-6-(biotinamido) hexanoate (Sulfo-NHS-LC-biotin) (Pierce, Rockford, Ill.) in PBS pH 8.0 for 1 h at 4° C. Cells were lysed in TNTE (50 mM Tris-HCl pH 7.4, 150 mM NaCl, 1% Triton-X100, 1 mM EDTA, and Protease Inhibitor Cocktail (Sigma). Aliquots of cell lysate were immunoprecipitated with anti-EGFR antibody (Sigma), and biotinylated surface proteins were captured on streptavidin-agarose beads. Proteins were separated by SDS-PAGE and probed with anti-EGFR (Santa Cruz Biotechnology, Santa Cruz, Calif.) antibody.

Results:

Identification of TGF-β Signaling Enhancers in a Cell-based Assay:

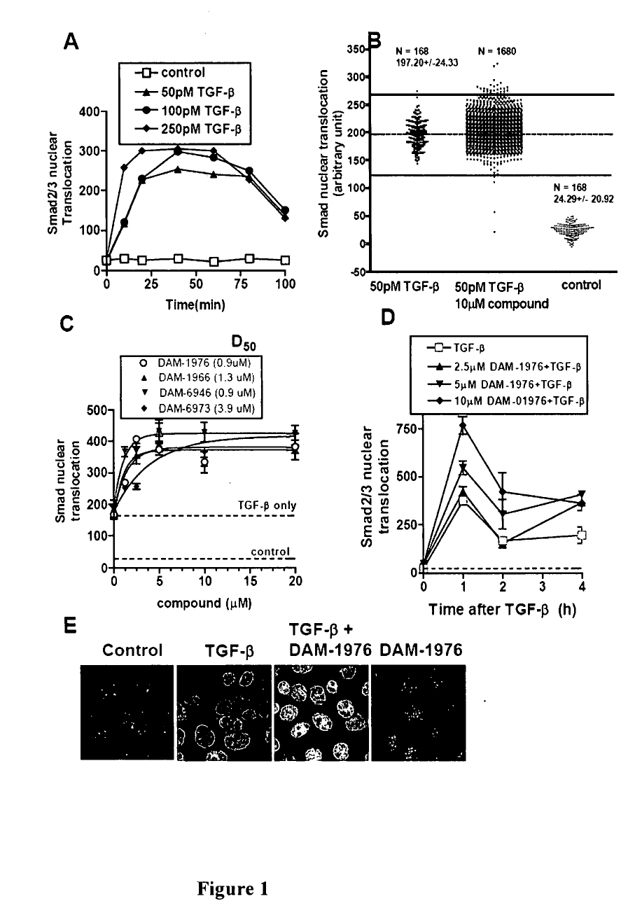

To identify chemical modifiers of TGF-β signaling, fluorescence imaging combined with an automated system were used to quantify cytoplasmic and nuclear Smad2/3. The algorithm calculates the difference between mean nuclear and cytoplasmic staining, and the method is more sensitive than conventional Western blotting (15). NMuMG mammary epithelial cells were grown in low serum conditions for 24 h, and stimulated with TGF-β1 to determine the optimal time and dose of cytokine for the screen. The cells displayed a 10-fold increase in nuclear Smad2/3 protein 20 min after addition of cytokine which was sustained until 50 min, then declined thereafter with an apparent half-life of ˜30 min (FIG. 1A). A dose of 50 pM TGF-β produced ˜80% saturation of Smad2/3 nuclear translocation. TGF-β did not stimulate Erk-p nuclear translocation, and EGF did not stimulate Smad2/3 nuclear translocation, demonstrating specificity of the assays (data not shown). The expectation was that chemical enhancers or inhibitors of Smad2/3 nuclear translocation would interact with a rate-limiting component of the signaling pathway.

Three compounds were initially identified that enhanced TGF-β1-induced nuclear translocation of Smad2/3 in the first 10,000 of the 50,000 compounds in the Maybridge Diversity set (FIG. 1B). The primary hit rate for enhancers and inhibitors was 0.1%, and the confirmed hit rate upon retesting was 0.03%. Five enhancers displayed D₅₀ values of 1-4 μM (FIG. 1C), and shared a core chemical structure of diethyl 2-(anilinomethylene)malonate (DAM) (Table 1). After an in silico structural screen of the rest of the library, a subset of plates was selected containing structurally related compounds, and two additional hits were identified (FIG. 1 and Table 1). DAM-1976, with an EC₅₀ of ˜1 μM, was selected for further characterization. DAM-1976 pre-treatment of NMuMG enhanced maximum nuclear Smad2/3 levels after TGF-β stimulation, and prolonged Smad2/3 nuclear residency (FIG. 1D). The effect of the DAM compounds on Smad2/3 phosphorylation following TGF-β stimulation was visualized in NMuMG cells by immunofluorescence microscopy (FIG. 1E).

DAM-1976 Sensitizes Cells to EGF and Autocrine TGF-β

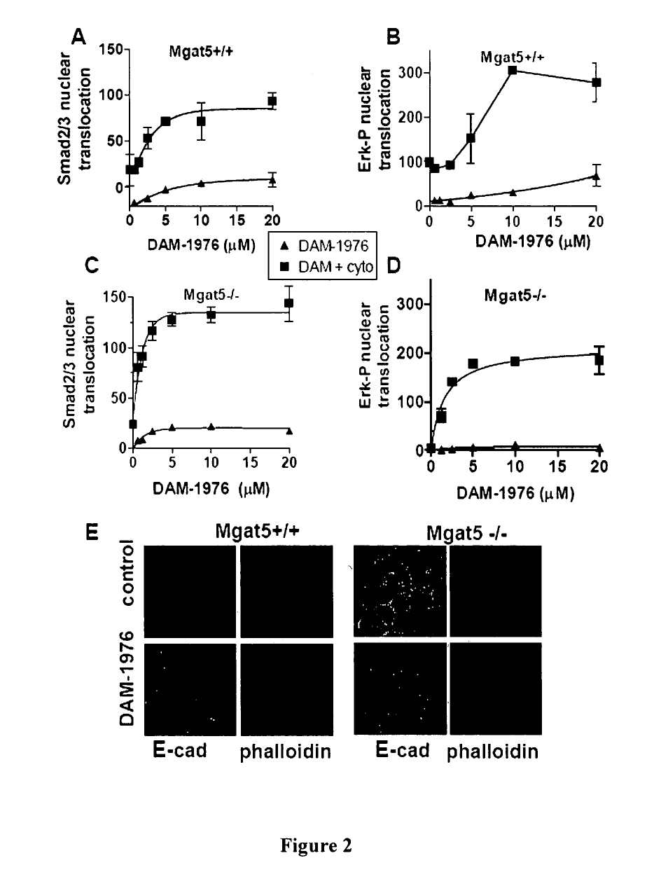

To determine whether DAM-1976 enhanced TGF-β signaling specifically, or sensitizes to multiple cytokines, mammary carcinoma cells were pre-treated with the drug and sensitivity to EGF and TGF-β was compared. DAM-1976 enhanced the sensitivity of cells to both cytokines with similar D₅₀ values (FIG. 2A-D). Since EGF and TGF-β signaling pathways are distinct, it appears that DAM-1976 acts on a target that broadly regulates cytokine sensitivity, possibly at the level of receptor availability. Consistent with this suggestion, DAM-1976 pre-treatment enhanced the sensitivity of Mgat5^{+/+}(2.6) and Mgat5^{-/-} (22.9) tumor cells to EGF and TGF-β. The Mgat5^{-/-}(22.9) cells are deficient in retention of cytokine receptors at the cell surface, and consequently poorly responsive to TGF-β and EGF (15). Pre-treatment of Mgat5^{-/-}(22.9) cells with DAM-1976 essentially rescues sensitivity to acute TGF-β and EGF (FIG. 2C,D). The initial characterization of the Mgat5^{-/-} defect demonstrated that cytokine signaling could be rescued by blocking endocytosis with a 1 h treatment of nystatin and K⁺ depletion (15). Nystatin disperses membrane cholesterol and disrupts caveolae-dependent endocytosis, while K⁺ depletion disrupts clathrin-assembly and coated-pits endocytosis. DAM-1976 may also disrupt receptor trafficking causing a sustained increase in surface levels that delivers greater intracellular activation of transducers.

Mgat5^{-/-}(22.9) tumor cells express the PyMT oncoprotein, which drives PI3K and Erk signaling, but the cells fail to undergo EMT as indicated by loss of E-cadherin in adhesion junctions. Transfection of the mutant cells with an Mgat5 expression vector restored autocrine TGF-β signaling and EMT (15). DAM-1976 treatment alone increased basal Smad2/3 nuclear localization indicating an enhancement of autocrine TGF-β signaling (FIG. 2A,C). Furthermore, DAM-1976 treatment of Mgat^{-/-}(22.9) cells induced loss of E-cadherin in adhesion junctions, and redistribution of microfilaments from a cortical to a basolateral position (FIG. 2E). However, the microfilaments were disorganized and punctuate, indicating shorter and branched forms of F-actin. DAM-1976 treatment of NMuMG cells also disrupted adhesion junction and increased basolateral F-actin, a phenotype similar to TGF-β treatment of these cells (data not shown).

DAM-1976 Slows Endosomal Trafficking and Prolongs Erk Activation

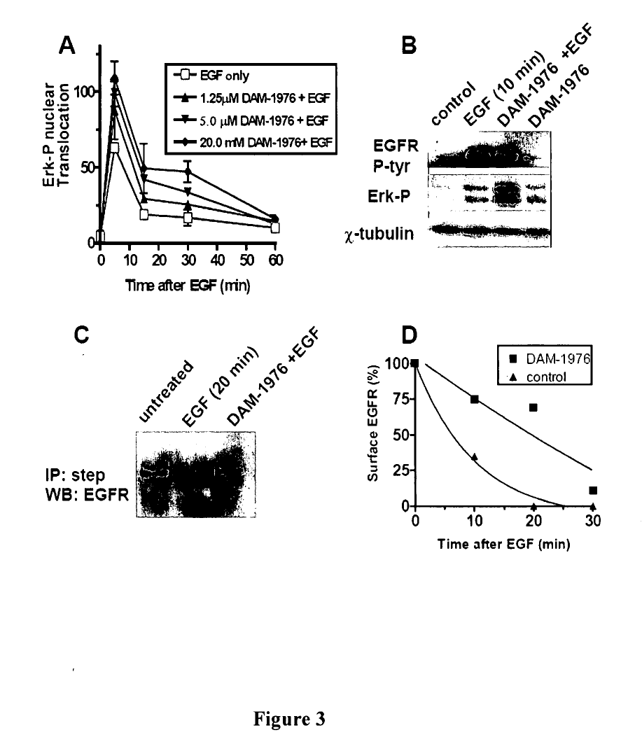

Next the possibility that DAM-1976 prolongs the activation state of signaling intermediates by slowing endocytosis and trafficking of signaling intermediates was explored. Maximal Erk-p translocation to the nucleus occurs ˜5 min after EGF addition and declines rapidly thereafter (FIG. 3A). DAM-1976 pre-treatment of Mgat5^{+/+}(2.6) cells enhanced peak levels of nuclear Erk-p and slowed the return to baseline (FIG. 3A). The levels of Erk-p were markedly higher in DAM-1976 pre-treated cells 10 min after EGF stimulation compared to untreated cells, indicating a prolongation of receptor signaling (FIG. 3B). Activation of the EGFR stimulates its internalization via clathrin coated-pits, and inactivation in the endososmes (20, 21). To measure the effect of DAM-1976 on EGFR endocytosis, Mgat5^{+/+}(2.6) cells were stimulated with EGF, and at times thereafter, surface labeled with sulfo-NHS-LC-biotin (FIG. 3C). Biotin-labeled EGFR was internalized at a 3 fold reduced rate in DAM-1976 pre-treated cells compared to untreated cells (FIG. 3D). This suggests that DAM-1976 delays endocytosis of activated EGFR, and thereby prolonged ligand-dependent receptor signaling. DAM-1976 induced a modest ligand-independent increase in nuclear Erk-p and Smad2/3 in Mgat5^{+/+}(2.6) cells, suggesting the compound sensitizes cells to autocrine stimulation.

DAM1976 Inhibits Microfilament Remodeling and Slows Tumor Cell Growth

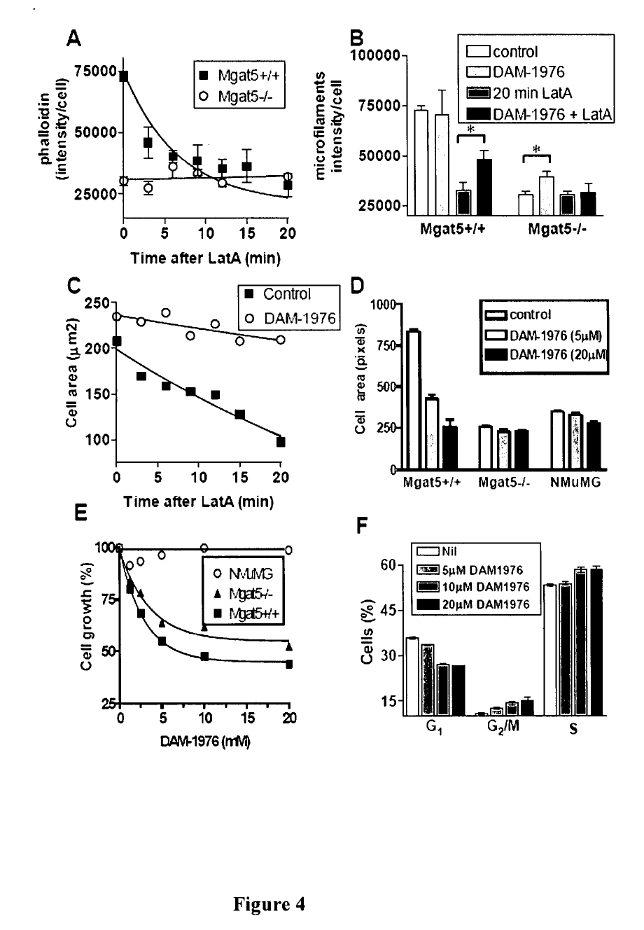

The microfilament assembly in Mgat5^{-/-}(22.9) is largely cortical, while that of Mgat5^{+/+}(2.6) cells span the substratum typical of the epithelial and mesenchymal phenotypes, respectively (FIG. 2E). In Mgat5^{-/-} cells, DAM-1976 treatment increased the basolateral microfilament content and reduced cortical stress fibers, producing a phenotype similar to Mgat5^{+/+}(2.6) cells, either with or with out drug (FIG. 2E). The basolateral microfilaments in Mgat5^{+/+}(2.6) cells disassemble rapidly in the presence of latrunculin-A (LatA), an inhibitor of actin re-polymerization, while LatA showed little effect in Mgat5^{-/-} tumor cells where basolateral filaments were constitutively very low (FIG. 4A). DAM-1976 inhibited LatA-dependent decay of basolateral microfilaments in Mgat5^{+/+}(2.6) cells, indicating that DAM-1976 slows de-polymerization of basal F-actin (FIG. 4B,C). Furthermore, DAM-1976 inhibited LatA-dependent retraction of Mgat5^{+/+}(2.6) cells on fibronectin by 70%, but only by 10% for Mgat5^{-/-}(22.9) cells, and 20% for non-malignant NMuMG cells (FIG. 4D).

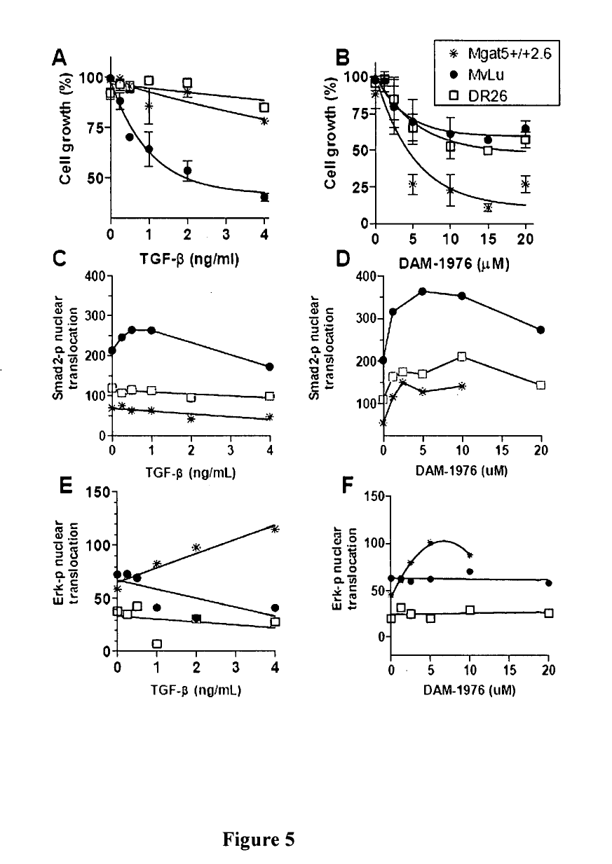

DAM-1976 reduced Mgat5^{+/+}(2.6) and Mgat5^{-/-}(22.9) tumor growth in tissue culture, but not that of non-transformed NMuMG epithelial cells under the same conditions (FIG. 4E). DAM-1976 treatment of Mgat5^{+/+}(2.6) displayed a dose-dependent increase in G2/M and S phase cells, indicating that the compound delays cell cycle progression (FIG. 4F). To further explore the mechanism of DAM-1976 dependent inhibition of carcinoma cell growth, cells were cultured for 48 h in the presence of increasing concentrations of either TGF-β or DAM-1976 and cell number and basal nuclear Erk-p and Smad2-p were measured. Mgat5^{+/+}(2.6) tumor cells were compared with non-transformed MvLu epithelial cells, and DR26 cells, an MvLu mutant cell line that lacks TβRII (22). TGF-β inhibited MvLu cell growth, but not that of DR26 or Mgat5^{+/+}(2.6) cells (FIG. 5A). In contrast, DAM-1976 reduced Mgat5^{+/+}(2.6) cell growth, while MvLu and DR26 cells were less severely inhibited, suggesting a different profile of growth suppression than TGF-β (FIG. 5B). Basal levels of nuclear Smad2-p were higher in MvLu cells, and increased slightly in the presence of TGF-β, while basal levels in Mgat5^{+/+}(2.6) cells and DR26 cells were lower, and not altered by culturing with TGF-β (FIG. 5C). Nuclear Smad2-p increased in all of the cell lines cultured with DAM-1976 including DR26 cells (FIG. 5D). This suggests DAM-1976 may enhance nuclear Smad2-p by a TGF-β independent mechanism, as well as sensitizing the cells to acute TGF-β signaling (FIG. 2). TGF-β and DAM-1976 enhanced nuclear Erk-p in Mgat5^{+/+}(2.6) cells, while a decline or no change was observed for MvLu and DR26 cells (FIG. 5E,F). Therefore, increases in both Erk-p and Smad2/3 activation accompany selective growth suppression of tumor cells by DAM-1976. The invasive phenotype requires activation of Ras/Erk, PI3 kinase/Akt growth pathways, and autocrine TGF-β/Smad2/3 signaling (8,9). However, hyper-activated Ras/Erk signaling becomes toxic in tumor cells (23), and Smad2/3 signaling is well known to promote cell cycle arrest (10, 11, 24). Therefore, it seems likely that changes in these and other signaling pathways in DAM-1976-treated carcinoma cells may contribute to growth suppression.

Discussion

Five compounds sharing a core structure of diethyl 2-(anilinomethylene) malonate (DAM) were isolated from the Maybridge Diversity Set chemical library, as enhancers of TGF-β dependent Smad2/3 nuclear translocation. These compounds access their targets in cells with D₅₀ values of 1-4 μM. Characterization of DAM-1976 activity suggests these compounds inhibit microfilament remodeling and enhance sensitivity to cytokines by prolonging the trafficking and activation of signaling intermediates.

Oncogenic signaling down-stream of receptor tyrosine kinases promotes vesicular trafficking and endocytosis (25). However, increases in membrane and microfilament remodelling in motile cells are balanced by mechanisms that protect cytokine receptors at the cell surface. In this regard, positive feedback from Ras/Erk signaling stimulates expression of the Mgat5 gene(17), which strengthens galectin-glycoprotein cross-linking and retains surface cytokine receptors in invasive tumor cells (15). Mgat5^{-/-}(22.9) cells are deficient in cell surface receptors, and blocking endocytosis restores their sensitivity to EGF and TGF-β. DAM-1976 pretreatment of Mgat5^{-/-}(22.9) tumor cells demonstrated a chemical rescue of this phenotype, and also enhanced responsiveness to EGF and TGF-β in Mgat5^{+/+}(2.6) cells. DAM-1976 enhanced autocrine TGF-β signaling as well as loss of E-cadherin in adhesion junctions. However, DAM-1976 did not rescue cell spreading in Mgat5^{-/-}(22.9) cells, but rather inhibited spreading of the wild type cells. This is consistent with the observations that although DAM-1976 enhances surface residency of receptors and sensitivity to cytokines, this may be secondary to the slowing of microfilament remodeling.

EGFR activation recruits Grb-2/Shc signaling complexes, but also Cbl, CIN85 and endophilins which stimulate endocytosis and inactivation of EGFR in endosomes (20, 21). In contrast, TGF-β does not stimulate TβR endocytosis, but rather receptors are internalized at bulk endocytosis rates (26). Therefore the target(s) of DAM-1976 are likely to be mediators of microfilament turnover and/or vesicular trafficking, which affect receptor trafficking both before and after ligand binding. Receptor internalization and vesicular trafficking requires the Rab family of small GTPases, and adaptor proteins (25). Activation of Arp2/3 complexes at the cell cortex stimulate actin polymerization required for endocytosis and cell shape changes (27). The cofilin/ADF protein accelerates actin filament turnover, and neural crest cells lacking n-cofilin display defects in F-actin bundling, cell polarization, and cell proliferation. The state of actin polymerization also regulates serum response factor (SRF) transcription factor activity and gene expression. Rho signaling induces F-actin polymerization and treadmilling, which reduces the pool of G-actin, releasing bound myocardin-related SRF coactivator (MAL) to translocate into the nucleus and activate (SRF)-dependent transcription (28). SRF forms regulatory complexes with Ets proteins and cooperates with Erk activation to stimulate early response genes. Agents that decrease actin treadmilling and MAL/SRF activity suppress early response gene expression. LatA blocks actin-treadmilling and increases G-actin levels, which retains MAL in the cytoplasm (29). DAM-1976 slows F-actin de-polymerization and actin treadmilling, which in a similar manner, may disrupt cell proliferation through changes in MAL/SRF activity.

Screens for chemical inhibitors of cytokine signaling such as EGFR/ErbB have yielded new anti-cancer agents (30). Herein a different approach is disclosed; noting that hyper-activation of intracellular signaling can be selectively toxic to tumor cells, chemical enhancers of cytokine signalling were identified. Tumor cells treated with enhancer DAM-1976 show stabilized microfilaments, slower endocytosis of EGFR, and increased sensitivity to EGF and TGF-β cytokines. Tumor cells cultured with DAM-1976 display increases in basal nuclear Erk-p and Smad2/3, which together with other changes in nuclear signal transducers may contribute to growth suppression. In summary, the DAM compounds represent a new pharmacophore, with a novel activity profile.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. The citation of any reference herein is not an admission that such reference is available as prior art to the instant invention.

TABLE 1
Pharmacophore of diethyl 2-(anilinomethylene) malonate compounds
identified in the screen for sensitizers of TGF-β1 signaling. The five compounds displayed
activity as enhancers of TGF-β induced Smad2/3 nuclear translocation with D₅₀ of 1-4 μM.

| Core Structure | | 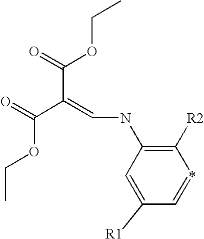 | | |
|---|---|---|---|---|
| ID | Variation | R1 | R2 | MW |
| DAM-1976 | * = C | 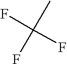 | 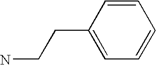 | 450.4 |
| DAM-1966 | * = C | 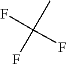 | 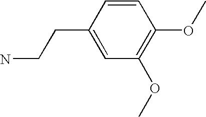 | 510.5 |
| DAM-06946 | * = C | H | 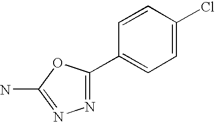 | 441.9 |
| DAM-06973 | * = C | H | 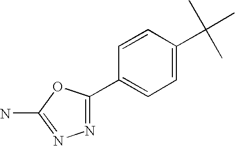 | 463.5 |
| DAM-09502 | * = O | H | 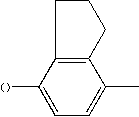 | 410.1 |

### Citations for Referenced Publications

1. Besson, A, Robbins, SM, and Yong, VW PTEN/MMACI/TEP1 in signal transduction and tumorigenesis. Eur J Biochem, 263: 605-11, 1999.
2. Chen, L, Zhang, W, Fregien, N, and Pierce, M The her-2/neu oncogene stimulates the transcription of N-acetylglucosaminyltransferase V and expression of its cell surface oligosaccharide products. Oncogene, 17: 2087-93, 1998.
3. Cui, W, Fowlis, D J, Bryson, S, et al. TGF-01 inhibits the formation of benign skin tumors, but enhances progression to invasive spindle carcinomas in transgenic mice. Cell, 86: 531-42, 1996.
4. Di Guglielmo, G M, Le Roy, C, Goodfellow, A F, and Wrana, J L Distinct endocytic pathways regulate TGF-β receptor signalling and turnover. Nature Cell Biol, 5: 410-21, 2003.
5. Granovsky, M, Fata, J, Pawling, J, et al. Suppression of tumor growth and metastasis in Mgat5-deficient mice. Nature Med, 6: 306-12, 2000.
6. Gurniak, C B, Perlas, E, and Witke, W The actin depolymerizing factor ncofilin is essential for neural tube morphogenesis and neural crest cell migration. Dev Biol, 278: 231-41, 2005.
7. Hirai, R and Fijita, T A human transforming growth factor-beta type II receptor that contains an insertion in the extracellular domain. Exp Cell Res, 223: 135-41, 1996.
8. Irani, K, Xia, Y, Zweier, J L, et al. Mitogenic signaling mediated by oxidants in ras-transformed fibroblasts. Science, 275: 1649-52, 1997.
9. Kang, R, Saito, H, Ihara, Y, et al. Transcriptional regulation of the acetylglucosaminyltranserase V gene in human bile duct carcinoma cells (HuCC-T1) is mediated by Ets-1. J Biol Chem, 271: 26706-12, 1996.
10. Lanzetti, L, Palamidessi, A, Areces, L, Scita, G, and Di Fiore, PP Rab5 is a signalling GTPase involved in actin remodelling by receptor tyrosine kinases. Nature, 429: 309-14, 2004.
11. Li, Z, Kim, ES, and Bearer, EL Arp2/3 complex is required for actin polymerization during platelet shape change. Blood, 99: 4466-74, 2002.
12. Lo, R S, Wotton, D, and Massague, J Epidermal growth factor signaling via Ras controls the Smad transcriptional co-repressor TGIF. EMBO J, 20: 128-36, 2001.
13. Lynch, T J, Bell, D W, Sordella, R, et al. Activating mutations in the epidermal growth factor receptor underlying responsiveness of non-small cell lung cancer to gefitinib. N Engl J Med, 350: 2129-39, 2004.
14. Massague, J How cells read TGF-beta signals. Nat Rev Mol Cell Biol, 1: 169-78, 2000.
15. Massague, J, Blain, S W, and Lo, R S TGF-β signaling in growth control, cancer, and heritable disorders. Cell, 103: 295-309, 2000.
16. Matsuura, I, Denissova, N G, Wang, G, et al. Cyclin-dependent kinases regulate the antiproliferative function of Smads. Nature, 430: 226-31, 2004.
17. Mehra, A and Wrana, J L TGF-beta and the Smad signal transduction pathway. Biochem Cell Biol, 80: 605-22, 2002.
18. Oft, M, Akhurst, R J, and Balmain, A Metastasis is driven by sequential elevation of H-ras and Smad2 levels. Nature Cell Biol, 4: 487-94, 2002.
19. Partridge, E A, Le Roy, C, Di Guglielmo, G M, et al. Regulation of cytokine receptors by Golgi N-glycan processing and endocytosis. Science, 306: 120-4, 2004.
20. Petrelli, A, Gilestro, G F, Lanzardo, S, et al. The endophilin-CIN85-Cbl complex mediates ligand-dependent downregulation of c-Met. Nature, 416 187-90, 2002.
21. Posern, G, Miralles, F, Guettler, S, and Treisman, R Mutant actins that stabilise F-actin use distinct mechanisms to activate the SRF coactivator MAL. Embo J, 23: 3973-83, 2004.
22. Seoane, J, Le, H V, Shen, L, Anderson, S A, and Massague, J Integration of Smad and forkhead pathways in the control of neuroepithelial and glioblastoma cell proliferation. Cell, 117: 211-23, 2004.
23. Soubeyran, P, Kowanetz, K, Szymkiewicz, I, Langdon, W Y, and Dikic, I Cbl-CIN85-endophilin complex mediates ligand-induced downregulation of EGF receptors. Nature, 416: 183-6, 2002.
24. Suh, N, Roberts, A B, Birkey Reffey, S, et al. Synthetic triterpenoids enhance transforming growth factor β/Smad signaling. Cancer Res, 63: 1371-6, 2003.
25. Thiery, J P Epithelial-mesenchymal transitions in development and pathologies. Curr Opin Cell Biol, 15: 740-6, 2003.
26. Valcourt, U, Kowanetz, M, Niimi, H, Heldin, C H, and Moustakas, A TGF-β and the Smad Signaling Pathway Support Transcriptomic Reprogramming during Epithelial-Mesenchymal Cell Transition. Mol Biol Cell, 2005.
27. Wakefield, L M and Roberts, A B. TGF-β signaling: positive and negative effects on tumorigenesis. 12: 22-9, 2002.
28. Wrana, J L, Attisano, L, Wieser, R, Ventura, F, and Massagu, J Mechanism of activation of the TGF-beta receptor. Nature, 370: 341-7, 1994.
29. Xiao, Y Q, Malcolm, K, Worthen, G S, et al. Cross-talk between ERK and p38 MAPK mediates selective suppression of pro-inflammatory cytokines by transforming growth factor-β. J Biol Chem, 277: 14884-93, 2002.
30. Zhang, J H, Chung, T D, and Oldenburg, K R A Simple Statistical Parameter for Use in Evaluation and Validation of High Throughput Screening Assays. J Biomol Screen, 4: 67-73, 1999.

What is claimed is:

1. A pharmaceutical composition comprising a compound of the formula I 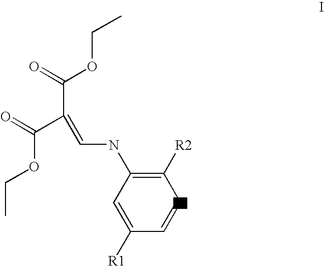
wherein * is ═C or ═O, R1 and R2 independently represent substituted or unsubstituted alkyl, alkenyl, alkynyl, alkylene, alkenylene, alkoxy, alkenyloxy, cycloalkyl, cycloalkenyl, aryl, aryloxy, arylalkoxy, aroyl, heteroaryl, heterocyclic, acyl, acyloxy, sulfonyl, sulfinyl, sulfenyl, amino, imino, azido, thiol, thioalkyl, thioalkoxy, thioaryl, nitro, ureido, cyano, halo, silyl, silyloxy, silythio, carboxyl, carbonyl, carbamoyl, carboxamide, carboxylic ester, phosphono, a substituted or unsubstituted aryl group fused to a cycloalkyl group, or a pharmaceutically acceptable salt thereof, and pharmaceutically acceptable carriers, excipients, and vehicles.

2. A pharmaceutical composition according to claim 1 wherein R1 represents:
a) hydrogen, substituted or unsubstituted alkyl, alkenyl, alkynyl, alkylene, alkoxy, acyl, acyloxy, sulfonyl, sulfinyl, sulfenyl, amino, imino, azido, thiol, thioalkyl, thioalkoxy, nitro, cyano, halo, silyl, silyloxy, silythio, carboxyl, carbonyl, carbamoyl, or carboxamide;
b) hydrogen, substituted or unsubstituted alkyl, alkenyl, alkoxy, amino, imino, azido, thiol, thioalkyl, nitro, cyano, halo, silyl, or carboxyl;
c) hydrogen or substituted or unsubstituted alkyl;
d) C₁-C₃ alkyl substituted with one or more halo; or
e) methyl substituted with one, two, or three halo.

3. A pharmaceutical composition according to claim 1 wherein R2 represents —NR15 wherein R15 is —R16R17 wherein R16 is substituted or unsubstituted C₁-C₆ alkyl, and R17 is substituted or unsubstituted aryl.

4. A pharmaceutical composition according to claim 3 wherein R16 is methyl, ethyl, or propyl, and R17 is substituted or unsubstituted phenyl.

5. A pharmaceutical composition according to claim 1 wherein R2 represents —NR15 wherein R15 is a substituted or unsubstituted heterocylic with 1 to 2 oxygen atoms and 1 to 3 nitrogen atoms or phenyl substituted with bromo, chloro, or substituted C₁-C₃-alkyl.

6. A pharmaceutical composition according to claim 1 wherein R2 represents:
a) substituted or unsubstituted aryl fused to a cycloalkyl;
b) substituted or unsubstituted phenyl fused with a cycloalkyl having 3 to 6 carbon atoms;
c) substituted or unsubstituted phenyl fused with a cyclopropyl, cyclobutyl or cyclopentyl;
d) phenyl fused to cyclopentyl wherein the phenyl is substituted with halo, alkyl, or oxy.

7. A pharmaceutical composition according to claim 1 wherein * is ═C, R1 represents hydrogen, substituted or unsubstituted alkyl, alkenyl, alkynyl, alkylene, alkoxy, acyl, acyloxy, sulfonyl, sulfinyl, sulfenyl, amino, imino, azido, thiol, thioalkyl, thioalkoxy, nitro, cyano, halo, silyl, silyloxy, silythio, carboxyl, carbonyl, carbamoyl, or carboxamide, and R2 represents —NR15 wherein R15 is —R16R17 wherein R16 is C₁-C₆ alkyl, and R17 is substituted or unsubstituted aryl.

8. A pharmaceutical composition according to claim 7 wherein R1 is substituted C₁-C₆ alkyl and R2 is NR15 wherein R15 is —R16R17 wherein R16 is C₁-C₆ alkyl and R17 is substituted or unsubstituted aryl.

9. A pharmaceutical composition according to claim 1 wherein R1 is methyl substituted with one, two or three fluoro and R2 is NR15 wherein R15 is —R16R17 wherein R16 is C₁-C₃ alkyl and R16 is aryl optionally substituted with hydroxyl, halo, alkyl, alkenyl, alkynyl, alkylene, alkoxy, acyl, acyloxy, sulfonyl, sulfinyl, sulfenyl, amino, imino, azido, thiol, thioalkyl, thioalkoxy, nitro, cyano, halo, silyl, silyloxy, silythio, carboxyl, carbonyl, carbamoyl, or carboxamide.

10. A pharmaceutical composition according to claim 1 wherein * is ═C, R1 represents hydrogen, substituted or unsubstituted alkyl, alkenyl, alkoxy, amino, imino, azido, thiol, thioalkyl, nitro, cyano, halo, silyl, or carboxyl and R2 represents —NR15 wherein R15 is an optionally substituted heterocylic with 1 to 2 oxygen atoms and 1 to 3 nitrogen atoms.

11. A pharmaceutical composition according to claim 10 wherein * is ═C, R1 represents hydrogen or substituted or unsubstituted alkyl and R2 represents —NR15 wherein R15 is substituted or unsubstituted 1,3,4-oxadiazolyl or R15 is an 1,3,4-oxadiazole substituted with optionally substituted alkyl, alkenyl, alkynyl, alkylene, alkoxy, acyl, acyloxy, sulfonyl, sulfinyl, sulfenyl, amino, imino, azido, thiol, thioalkyl, thioalkoxy, nitro, cyano, halo, silyl, silyloxy, silythio, carboxyl, carbonyl, carbamoyl, or carboxamide.

12. A pharmaceutical composition according to claim 1 wherein * is ═O, R1 represents hydrogen, substituted or unsubstituted alkyl or alkoxy, sulfonyl, sulfinyl, sulfenyl, amino, imino, azido, thiol, nitro, cyano, halo, silyl, carboxyl, carbonyl, or carbamoyl, and R2 is a substituted or unsubstituted aryl fused to a cycloalkyl having 3 to 6 carbon atoms.

13. A pharmaceutical composition according to claim 12 wherein R1 represents hydrogen or substituted or unsubstituted alkyl and R2 is substituted or unsubstituted phenyl fused to a cycloalkyl having 3 to 6 carbon atoms.

14. A pharmaceutical composition according to claim 13 wherein R2 is substituted phenyl fused to cyclopropyl or R2 is phenyl substituted with halo, ═O, alkyl, or alkoxy, fused to cyclopropyl at positions 2 to 3.

15. A pharmaceutical composition according to claim 1 wherein R1 is —NH—CH₂CH₂-Ph, R2 is —CF₃, and/or, (ii) R1 is an oxadiazolyl substituted with chlorophenyl, when R2 is hydrogen.

16. A pharmaceutical composition according to claim 1 wherein a compound of the formula I is a compound listed in Table 1.

17. A pharmaceutical composition according to claim 1 wherein a compound of the formula I has a structure of a diethyl 2 (anilinomethylene) malonate compound or derivative thereof.

18. A method for treating a proliferative disease comprising administering to a subject in need of such treatment a composition according to claim 1.

19. A method according to claim 18 wherein the proliferative disease is cancer, tumor invasion, tumor growth, and/or tumor metastasis.

20. A method of enhancing TGFβ dependent Smad2/3 translocation in a subject in need thereof comprising administering to the subject a therapeutically effective amount of a composition according to claim 1.
